SEQUENCE LISTING (6 sequences)

SEQ ID NO: 1 - DNA, 29 bp, Artificial Sequence
  artifically synthesized sequence
ccgaggtcgaaacgtacgttctctctatc

SEQ ID NO: 2 - DNA, 32 bp, Artificial Sequence
  artifically synthesized sequence
tgacaggattggtcttgtctttagccattcca

SEQ ID NO: 3 - DNA, 22 bp, Artificial Sequence
  artifically synthesized sequence
atctcggctttgagggggcctg

SEQ ID NO: 4 - DNA, 22 bp, Artificial Sequence
  artifically synthesized sequence
tgctaagactccccaccgtaac

SEQ ID NO: 5 - DNA, 24 bp, Artificial Sequence
  artifically synthesized sequence
ggatttttgcaggattgtttatga

SEQ ID NO: 6 - DNA, 16 bp, Artificial Sequence
  artifically synthesized sequence
cacttgccctgcacca

(19) United States
(12) Patent Application Publication
(10) Pub. No.: US 2024/0271184 A1
(43) Pub. Date: Aug. 15, 2024

(54) LOW PH COMPOSITION AND METHOD FOR STABILIZING NUCLEIC ACIDS IN BIOLOGICAL SAMPLES

(71) Applicant: DNA Genotek Inc., Kanata (CA)

(72) Inventors: Brice Georges Le Francois, Ottawa (CA); Alaya Mikalauskas, Carleton Place (CA); Bitapi Ray, Ottawa (CA); Rafal Michal Iwasiow, Ottawa (CA)

(21) Appl. No.: 18/568,538

(22) PCT Filed: Jun. 8, 2022
(86) PCT No.: PCT/CA2022/050919

§ 371 Date: Dec. 8, 2023

Related U.S. Application Data
(60) Provisional application No. 63/208,212, filed on Jun. 8, 2021.

(51) Int. Cl. C12Q 1/6806

(52) U.S. Cl. CPC C12Q 1/6806

(57) ABSTRACT

An aqueous composition for stabilizing nucleic acid contained in a biological sample at ambient temperature is provided. The aqueous composition comprises: (i) a denaturing agent selected from sodium dodecyl sulphate (SDS) or a guanidinium salt; (ii) aurintricarboxylic acid (ATA); and (iii) at least one of a chelating agent and a buffering agent; wherein the composition has a pH of 4.9 or less. A method of stabilizing nucleic acid contained in a biological sample at ambient temperature is also provided, wherein the method comprises the steps of: a) obtaining a biological sample; b) contacting the biological sample with the above-noted aqueous composition to form a mixture; c) homogenizing the mixture of (b) to form a homogeneous mixture; and d)storing the homogeneous mixture at ambient temperature.

### FIELD OF THE INVENTION

The present invention pertains to an aqueous composition and method for stabilizing nucleic acid contained in a biological sample at ambient temperature.

### BACKGROUND

The realization that the human body is actually a “super-organism” that contains more microbial cells than all cells in the mammalian body has revolutionized our thinking about human systems' biology and the management of health and disease at the systemic level (Ley et al., 2006). The importance of the microbiome in human health and disease is becoming increasingly clear (Choo et al., 2015; Koenig et al., 2010; Belstrom, 2020). Compelling evidence now exists that bacterial colonization plays a central role in health and disease (Dethlefsen et al., 2007) such as the development and regulation of the host immunity (Hansen et al., 2014; Clarke et al., 2010; Koboziev et al., 2014), metabolism (Yano et al., 2015), and even the gut-brain axis (Carabotti et al., 2015). Disruption of these homeostatic roles through perturbation of the normal microbial community has been associated with a wide range of pathological conditions; including obesity (Ley et al., 2006; Backhed et al., 2004), autoimmune diseases (Scher et al., 2013), chronic gastrointestinal (GI) inflammatory diseases (Schaubeck et al., 2015; Hedin et al., 2015; Koboziev et al., 2014), type I and II diabetes (Kostic et al., 2015; Qin et al., 2012) and carcinogenesis (Schulz et al., 2014; Feng et al., 2014).

Next generation sequencing technologies have provided powerful tools to study associations between human microbiome and disease (Flores et al., 2015). However, the results from high-throughput sequencing can be biased by numerous factors. To advance the field of human microbiome research, validated specimen collection methods are needed, that capture as closely as possible, the true in vivo state of the sample and account for any technical variation that can be introduced. Specimen collection methods must be acceptable to participants and tolerant of suboptimal field conditions. If optimal means of collection or storage at—80° C. are not possible, these processing steps can introduce a systematic bias in biological samples. Hence, it is essential to minimize possible artifacts by developing and validating collection methods that can be easily implemented for both clinical use and for large field-based epidemiologic studies (Flores et al., 2015).

Sample collection procedures represent one of the first crucial steps that ensure accuracy, integrity and stability of the collected material (Panek et al., 2018). Numerous reports showed the importance of the collection procedures on sample quality (Cardona et al., 2012; Choo et al., 2015; Guo et al., 2016). Accuracy of results is increased by prompt sample processing (within 2-3 hours), immediate stabilization (Choo et al., 2015; Anderson et al., 2016) or appropriate storage conditions (Cardona et al., 2012; Choo et al., 2015; Guo et al., 2016; Carroll et al., 2012; Fouhy et al., 2015). Cold chain handling of specimens cannot always be guaranteed from the site of sample collection to the site of sample processing. Specimens collected in the field may spend various amounts of time at ambient temperature, followed by shipment on frozen gel packs (4° C.) or dry ice to a central laboratory for processing and storage. If immediate storage at −80° C. is not possible, a preservative is needed to prevent differential growth of bacteria or changes in analytes of interest that can occur during typical delays encountered in field studies. An ideal preservative would conserve the stability of total nucleic acids, both DNA and RNA, in the biospecimen at ambient temperature. Ambient temperature storage and shipment of samples would greatly facilitate and standardize studies by enabling easy collection in a participant's home, avoiding inconsistent sample handling and storage, and reducing shipping costs.

The structure and function of the human microbiome is currently largely inferred from metagenomics and metatranscriptomic analyses. Recovery of intact DNA and RNA is therefore essential for these studies. Different storage conditions can alter i) the stability of the microbial communities in the sample and ii) affect the quality of extracted nucleic acids. Microbial profiles and metabolic activity will change quickly if samples are kept at room temperature. Similarly, RNA and DNA degradation will also quickly occur when samples are held at ambient temperature or defrosted for a short period (1 hour) before nucleic acid extraction. Conditions that affect DNA and RNA integrity will alter the relative abundance of taxa in downstream bacterial community analysis (Cardona et al., 2012).

Nucleases are a large group of enzymes and ribozymes that are essential to many cellular processes, such as DNA replication and repair, RNA maturation, defense against pathogens, programmed cell death and RNA/DNA decay (Yang, 2011). Being such a broad and complex family their classification is quite difficult. They can be divided based on substrate specificity (DNAses vs RNAses), although many nucleases can process both DNA and RNA, or alternatively as endo- or exo-nucleases based on their mode of action and reaction by-products (Zhang and Reha-Krantz, 2013). Perhaps more importantly, nucleases can be divided into three major classes based on their catalytic mechanism and requirement for metal ions (1 or 2 metal ion-dependent vs metal-ion-independent nucleases) (Dupureur, 2008; Yang, 2011).

The major human nucleases tend to be fairly well characterized, with two main DNAses families (DNAse 1 and DNAse 2) based on requirement for a metal-ion for catalysis (Keyel, 2017). Similarly, various classes of RNAses are found in human cells such as RNAse H, RNAse T2 or the vertebrate specific RNAse A family (Sorrentino, 2010; Thorn et al. 2012). Bacterial nucleases on the other hand, remain much less characterized, with most of the work primarily focusing on two model organisms, Escherichia coli and Bacillus subtilis. E. coli alone is known to express 17 DNA exonucleases (Lovett, 2011), 9 RNA endonucleases and 7 RNA exonucleases (Bechhofer and Deutscher, 2019). B. subtilis also expresses a large number of nucleases, many of which are not found in E. coli (Condon, 2003). For example, the main RNA degradation enzyme in B. subtilis and firmicutes is RNAse Y, instead of RNAse E in E. coli (Commichau et al., 2009). In addition to traditional nucleases, most bacteria also express toxins, such as RelB or YoeB, that are activated under stressful conditions to block protein translation by quickly degrading ribosome-associated mRNAs (Pavelich et al. 2019). While E. coli and B. subtilis nucleases are starting to be fairly well understood, future work will likely lead to the discovery of a plethora of new enzymes in less studied bacterial species. Importantly, cataloging the entire spectrum of nucleases that can be present in complex biological specimens, such as human microbiome samples, is virtually an impossible task given that such samples can contain hundreds of bacterial species as well as human cells.

Inhibition or inactivation of nucleases is critical to maintain the integrity of nucleic acids in biological samples. Many strategies have been used over the years and include incubation of the samples with strong denaturing agents (e.g. guanidine salts or detergents) or incubation of the sample with proteases to inactivate proteins. Addition of chelating agents to samples is also an efficient way to inhibit the activity of any nuclease that requires metal ions for catalysis (Barra et al., 2015). However, nucleases don't always require metal ions for activity and can be extremely difficult to inactivate. For example, members of the RNAse A family are highly stable nucleases that can readily re-fold following denaturation. Inhibition of such types of nuclease can be achieved by addition of ribonuclease inhibitor proteins (Kim et al., 1999), treating the sample with the alkylating agent diethyl pyrocarbonate (DEPC) (Wolf et al., 1970) or reducing the disulfide bonds essential to their conformation with DTT or β-mercaptoethanol (Chen et al., 2004). Nevertheless, all of these approaches have pitfalls (toxicity, cost, reversibility of the inactivation) and they are unable to completely prevent RNAse activity (Blumberg, 1987). Bacterial nucleases are less characterized than their mammalian counterparts and little is known about their activity or their potential inhibitors and/or regulation. Several compounds have been shown to inhibit bacterial RNAses, such as poly(vinylsulfonic acid) (PVSA), aminoglycosides or toluidine blue (Earl et al., 2018; Mikkelsen et al., 1999; Wu et al., 2016). Small molecules inhibitors of the DEDDh or RNAse E family have also been described (Huang et al. 2016; Kime et al., 2015), but most of these are highly specific and presumably only active against specific classes of nucleases that share a similar conserved structure. Moreover, mammalian and bacterial nucleases can be active across a broad pH range (Blumberg, 1987; Condon, 2003) making their inhibition even more challenging. To this day, no method to efficiently inhibit the large number of nucleases present in complex biological samples has been described.

There is a need for compositions and methods for stabilizing nucleic acid contained in a biological sample at ambient temperature

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In one aspect, there is provided a method of stabilizing nucleic acid contained in a biological sample at ambient temperature comprising the steps of: a) obtaining a biological sample; b) contacting the biological sample with an aqueous composition to form a mixture, wherein the aqueous composition comprises: (i) a denaturing agent selected from sodium dodecyl sulphate (SDS), lithium dodecyl sulphate, or a guanidinium salt; (ii) aurintricarboxylic acid (ATA), or a salt thereof; and (iii) at least one of a chelating agent and a buffering agent; wherein the composition has a pH of 4.9 or less; c) homogenizing the mixture of (b) to form a homogeneous mixture; and d) storing the homogeneous mixture at ambient temperature.

In another aspect, there is provided an aqueous composition for stabilizing nucleic acids contained in a biological sample at ambient temperature, comprising: (i) a denaturing agent selected from sodium dodecyl sulphate (SDS), lithium dodecyl sulphate, or a guanidinium salt; (ii) aurintricarboxylic acid (ATA), or a salt thereof; and (iii) at least one of a chelating agent and a buffering agent; wherein the composition has a pH of 4.9 or less.

### BRIEF DESCRIPTION OF THE FIGURES

For a better understanding of the present invention including the progression of development to get to the end product, reference is made to the following description which is to be used in conjunction with the accompanying drawings, where:

FIG. 1A illustrates results of Agilent 4200 Tapestation analysis, showing stability of RNA in saliva stored in the present composition with increasing concentrations of ATA (donor 1).

FIG. 1B illustrates results of Agilent 4200 Tapestation analysis, showing stability of RNA in saliva stored in the present composition with increasing concentrations of ATA (donor 2).

FIG. 1C illustrates results of Agilent 4200 Tapestation analysis, showing stability of genomic DNA in saliva stored in the present composition with increasing concentrations of ATA (donor 1).

FIG. 1D illustrates results of Agilent 4200 Tapestation analysis, showing stability of genomic DNA in saliva stored in the present composition with increasing concentrations of ATA (donor 2).

FIG. 1E illustrates results of Agilent 4200 Tapestation analysis, showing stability of RNA in saliva stored in the present composition with increasing concentrations of PAAc with or without ATA (donor 1).

FIG. 1F illustrates results of Agilent 4200 Tapestation analysis, showing stability of RNA in saliva stored in the present composition with increasing concentrations of PAAc with and without ATA (donor 2).

FIG. 1G illustrates results of Agilent 4200 Tapestation analysis, showing stability of genomic DNA in saliva stored in the present composition with increasing concentrations of PAAc with and without ATA (donor 1).

FIG. 1H illustrates results of Agilent 4200 Tapestation analysis, showing stability of genomic DNA in saliva stored in the present composition with increasing concentrations of PAAc with and without ATA (donor 2).

FIG. 2A illustrates results of Agilent 4200 Tapestation analysis, showing impact of pH on exogenous RNA stability in saliva (donor 1).

FIG. 2B illustrates results of Agilent 4200 Tapestation analysis, showing impact of pH on exogenous RNA stability in saliva (donor 2).

FIG. 2C illustrates results of Agilent 4200 Tapestation analysis, showing endogenous stool RNA stability and integrity following storage at room temperature and low pH for 9 days in the present composition.

FIG. 2D illustrates results of Agilent 4200 Tapestation analysis, showing endogenous stool DNA stability and integrity following storage at room temperature and low pH for 9 and 16 days in the present compositions.

FIG. 3A illustrates results of Agilent 4200 Tapestation analysis, showing RNA stability in saliva in response to different classes of detergents (donor 1).

FIG. 3B illustrates results of Agilent 4200 Tapestation analysis, showing RNA stability in saliva in response to different classes of detergents (donor 2).

FIG. 3C illustrates results of Agilent 4200 Tapestation analysis, showing RNA stability in saliva in response to different classes of detergents (donor 3).

FIG. 3D illustrates results of Agilent 4200 Tapestation analysis, showing DNA stability in saliva in response to different classes of detergents (donor 1).

FIG. 3E illustrates results of Agilent 4200 Tapestation analysis, showing DNA stability in saliva in response to different classes of detergents (donor 2).

FIG. 3F illustrates results of Agilent 4200 Tapestation analysis, showing RNA stability in stool in response to different classes of detergents (donor 1).

FIG. 3G illustrates results of Agilent 4200 Tapestation analysis, showing RNA stability in stool in response to different classes of detergents (donor 2).

FIG. 3H illustrates results of Agilent 4200 Tapestation analysis, showing DNA stability in stool in response to different classes of detergents (donor 1).

FIG. 3I illustrates results of Agilent 4200 Tapestation analysis, showing DNA stability in stool in response to different classes of detergents (donor 2).

FIG. 3J illustrates results of Agilent 4200 Tapestation analysis, showing DNA stability in stool in response to different classes of detergents (donor 3).

FIG. 4A illustrates results of Agilent 4200 Tapestation analysis, showing the effect of increasing concentrations of CDTA on spiked RNA stability in stool (donor 1).

FIG. 4B illustrates results of Agilent 4200 Tapestation analysis, showing the effect of increasing concentrations of CDTA on spiked RNA stability in stool (donor 2).

FIG. 4C illustrates results of Agilent 4200 Tapestation analysis, showing the effect of increasing concentrations of CDTA on spiked RNA stability in stool (donor 3).

FIG. 4D illustrates results of Agilent 4200 Tapestation analysis, showing the effect of CDTA on endogenous genomic DNA stability in stool (donor 1, 2 and 3).

FIG. 4E illustrates results of Agilent 4200 Tapestation analysis, showing the effect of CDTA and EDTA on endogenous RNA stability in stool samples (donor 1).

FIG. 4F illustrates results of Agilent 4200 Tapestation analysis, showing the effect of CDTA and EDTA on endogenous RNA stability in stool samples (donor 2).

FIG. 4G illustrates results of Agilent 4200 Tapestation analysis, showing the effect of CDTA and EDTA on endogenous RNA stability in stool samples (donor 3).

FIG. 4H illustrates results of Agilent 4200 Tapestation analysis, showing the effect of CDTA and EDTA on endogenous DNA stability in stool samples (donor 1).

FIG. 4I illustrates results of Agilent 4200 Tapestation analysis, showing the effect of CDTA and EDTA on endogenous DNA stability in stool samples (donor 2).

FIG. 4J illustrates results of Agilent 4200 Tapestation analysis, showing the effect of CDTA and EDTA on endogenous DNA stability in stool samples (donor 3).

FIG. 5A illustrates results of Agilent 4200 Tapestation analysis, showing the effect of buffering agents on endogenous RNA (left) and DNA (right) stability in stool samples (donor 1).

FIG. 5B illustrates results of Agilent 4200 Tapestation analysis, showing the effect of buffering agents on endogenous RNA (right) and DNA (left) stability in stool samples (donor 2).

FIG. 5C illustrates results of Agilent 4200 Tapestation analysis, showing the effect of buffering agents on endogenous RNA (left) and DNA (right) stability in stool samples (donor 3).

FIG. 6A illustrates results of Agilent 4200 Tapestation analysis, showing the effect of salts on spiked RNA stability in saliva samples (donor 1).

FIG. 6B illustrates results of Agilent 4200 Tapestation analysis, showing the effect of salts on spiked RNA stability in saliva samples (donor 2).

FIG. 6C illustrates results of Agilent 4200 Tapestation analysis, showing the effect of salts on spiked RNA stability in saliva samples (donor 3).

FIG. 6D illustrates results of Agilent 4200 Tapestation analysis, showing the effect of salts on endogenous DNA stability in saliva samples (donor 1).

FIG. 6E illustrates results of Agilent 4200 Tapestation analysis, showing the effect of salts on endogenous DNA stability in saliva samples (donor 2).

FIG. 6F illustrates results of Agilent 4200 Tapestation analysis, showing the effect of salts on endogenous RNA stability in stool samples (donor 1).

FIG. 6G illustrates results of Agilent 4200 Tapestation analysis, showing the effect of salts on endogenous RNA stability in stool samples (donor 2).

FIG. 6H illustrates results of Agilent 4200 Tapestation analysis, showing the effect of salts on endogenous RNA stability in stool samples (donor 3).

FIG. 6I illustrates results of Agilent 4200 Tapestation analysis, showing the effect of salts on endogenous DNA stability in stool samples (donor 1).

FIG. 6J illustrates results of Agilent 4200 Tapestation analysis, showing the effect of salts on endogenous DNA stability in stool samples (donor 2).

FIG. 6K illustrates results of Agilent 4200 Tapestation analysis, showing the effect of salts on endogenous DNA stability in stool samples (donor 3).

FIG. 7A illustrates results of Agilent 4200 Tapestation analysis, showing the effect of pH and nuclease inhibitors on exogenous RNA stability in saliva (donor 1) collected into GTC-based preservative.

FIG. 7B illustrates results of Agilent 4200 Tapestation analysis, showing the effect of pH and nuclease inhibitors on exogenous RNA stability in saliva (donor 2) collected into GTC-based preservative.

FIG. 7C illustrates results of Agilent 4200 Tapestation analysis, showing the effect of pH and nuclease inhibitors on exogenous RNA stability in saliva (donor 3) collected into GTC-based preservative.

FIG. 7D illustrates results of Agilent 4200 Tapestation analysis, showing the effect of pH and nuclease inhibitors on exogenous RNA stability in saliva (donor 1) collected into GuHCl-based preservative.

FIG. 7E illustrates results of Agilent 4200 Tapestation analysis, showing the effect of pH and nuclease inhibitors on exogenous RNA stability in saliva (donor 2) collected into GuHCl-based preservative.

FIG. 7F illustrates results of Agilent 4200 Tapestation analysis, showing the effect of pH and nuclease inhibitors on exogenous RNA stability in saliva (donor 3) collected into GuHCl-based preservative.

FIG. 8A is a chart illustrating results of a nuclease detection assay in guanidinium thiocyanate-based chemistries mixed with saliva (1:1 ratio).

FIG. 8B is a chart illustrating results of a nuclease detection assay in guanidinium hydrochloride-based chemistries mixed with saliva (1:1 ratio).

FIG. 9A illustrates results of Agilent 4200 Tapestation analysis, showing endogenous RNA (pellet and supernatant fractions) in stool samples mixed at different ratios with the compositions listed in Table 4 (donor 1).

FIG. 9B illustrates results of Agilent 4200 Tapestation analysis, showing endogenous RNA (pellet and supernatant fractions) in stool samples mixed at different ratios with the compositions listed in Table 4 (donor 2).

FIG. 9C is a chart illustrating results of a nuclease detection assay in stool samples mixed at different ratios with the compositions listed in Table 4 (donor 1).

FIG. 9D is a chart illustrating results of a nuclease detection assay in stool samples mixed at different ratios with the compositions listed in Table 4 (donor 2).

FIGS. 10A-D illustrate results of Agilent 4200 Tapestation analysis, showing endogenous RNA and DNA stability in the supernatant and pellet fractions of stool samples from 3 donors stored in the present compositions for 1 day at room temperature.

FIG. 10E is a chart illustrating results of a nuclease detection assay in stool samples from 3 donors stored in the present compositions for one day at room temperature.

FIG. 11A illustrates results of Agilent 4200 Tapestation analysis, showing endogenous stool RNA stability following storage at room temperature for 0, 7 or 14 days in the current formulation compared to RNA extracted from the raw sample at baseline.

FIG. 11B illustrates results of Agilent 4200 Tapestation analysis, showing endogenous stool DNA stability following storage at room temperature for 0, 7 or 14 days in the current formulation compared to DNA extracted from the raw sample at baseline.

FIG. 11C is a chart illustrating metatranscriptomic profile stability (family level) of stool samples stored in the present formulation for 0, 7 or 14 days compared to the raw sample at baseline.

FIG. 12A illustrates results of Agilent 4200 Tapestation analysis showing endogenous RNA stability in stool samples from 12 donors at T0 when collected into the present composition.

FIG. 12B illustrates results of Agilent 4200 Tapestation analysis showing endogenous RNA stability in stool samples from 12 donors at T12 when collected into the present composition and stored at room temperature.

FIG. 12C illustrates results of Agilent 4200 Tapestation analysis showing endogenous DNA stability in stool samples from 12 donors at T0 when collected into the present composition.

FIG. 12D illustrates results of Agilent 4200 Tapestation analysis showing endogenous DNA stability in stool samples from 12 donors at T12 when collected into the present composition and stored at room temperature.

FIG. 12E illustrates results of Agilent 4200 Tapestation analysis showing exogenous RNA stability in stool samples from 12 donors at T1 and T3 when collected into the present composition and stored at 37° C.

FIG. 12F illustrates results of Agilent 4200 Tapestation analysis showing endogenous RNA stability in stool samples from 12 donors at T5 when collected into the present composition and subjected to three cycles of freeze/thaw at the indicated temperature.

FIG. 13A illustrates results of Agilent 4200 Tapestation analysis showing endogenous RNA stability in stool samples from 3 infants at T0 and T7 when collected into the present composition.

FIG. 13B illustrates results of Agilent 4200 Tapestation analysis showing endogenous DNA stability in stool samples from 3 infants at T0 and T7 when collected into the present composition.

FIG. 14A illustrates results of Agilent 4200 Tapestation analysis showing endogenous RNA stability in saliva samples from 3 representative donors following storage at room temperature for 21 or 60 days in the present composition compared to RNA extracted at baseline.

FIG. 14B illustrates human and viral mRNA stability as determined by RT-qPCR analysis following storage at room temperature for 21 or 60 days in the present composition compared to baseline.

FIG. 14C is a chart illustrating bacterial DNA and RNA profile stability (16S amplicon sequencing—genus level) of saliva samples from 2 representative donors stored in the present composition for 21 or 60 days compared to the sample at baseline.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “sample” as used herein will be understood to mean any specimen that potentially contains a substance of interest, in particular a nucleic acid, and optionally a protein or other biomolecules of interest. The term “sample” can encompass a solution, such as an aqueous solution, cell, tissue, biopsy, powder, or population of one or more of the same. The sample can be a biological sample, such as saliva, sputum, buccal swab sample, serum, plasma, blood, buffy coat, pharyngeal, nasal/nasal pharyngeal or sinus swabs or secretions, throat swabs or scrapings, urine, mucous, feces/stool/excrement, rectal swabs, lesion swabs, chyme, vomit, gastric juices, pancreatic juices, gastrointestinal (GI) tract fluids or solids, semen/sperm, urethral swabs and secretions, cerebral spinal fluid, products of lactation or menstruation, egg yolk, amniotic fluid, aqueous humour, vitreous humour, cervical secretions or swabs, vaginal fluid/secretions/swabs or scrapings, bone marrow samples and aspirates, pleural fluid and effusions, sweat, pus, tears, lymph, bronchial or lung lavage or aspirates, peritoneal effusions, cell cultures and cell suspensions, connective tissue, epithelium, epithelial swabs and smears, mucosal membrane, muscle tissue, placental tissue, biopsies, exudates, organ tissue, nerve tissue, hair, skin, or nails, wherein samples of the foregoing may be obtained from for example, a vertebrate, including a mammal. A mammal can be, for example, a human, a non-human primate, cattle (such as cow, goat, or sheep), as well as a dog, cat, horse, etc.

In one embodiment, the biological sample is a fecal sample and the subject is a mammal. In another embodiment, the biological sample is a fecal sample and the subject is a human. In one embodiment, the biological sample is a saliva sample and the subject is a mammal. In another embodiment, the biological sample is a saliva sample and the subject is a human.

Other types of biological samples can include plants, plant extracts, algae, soil samples, sewage, wastewater, water, environmental samples, foodstuff, cattle feed, fish feed, animal feed, swabs of contaminated or potentially infectious surfaces or equipment (e.g. meat processing surfaces), swabs from ‘touch’ surfaces in hospitals, nursing homes, outpatient facilities, medical institutions, or the like. In still other embodiments, the biological sample is selected from a soil sample, a sewage sample, a wastewater sample, or a water sample, any of which may be contaminated with feces.

The term “nuclease-rich donor” as used herein with reference to samples refers to a sample that contains either higher levels of nucleases or a greater diversity of nucleases relative to the general population, and as such is a sample where stabilization of nucleic acids is more challenging.

The term “ambient temperature” as used herein refers to a range of temperatures that could be encountered by the mixture of the biological sample (e.g. feces or saliva sample) and the aqueous composition described herein from the point of collection, during transport (which can involve relatively extreme temperatures, albeit usually for shorter periods of time (e.g. <5 days)), as well as during prolonged storage prior to analysis. In one embodiment, the ambient temperature is ranging from about −20° C. to about 50° C. In another embodiment, the ambient temperature is room temperature (RT) and ranges from about 15° C. to about 25° C.

The term “chelator” or “chelating agent” as used herein will be understood to mean a chemical that will form a soluble, stable complex with certain metal ions (e.g., Ca²⁺ and Mg²⁺), sequestering the ions so that they cannot normally react with other components, such as deoxyribonucleases (DNases) or ribonucleases (RNAses) or endonucleases (e.g. type I, II and III restriction endonucleases) and exonucleases (e.g. 3′ to 5′ exonuclease), enzymes which are abundant in various biological samples. In the present composition, chelating agent(s) participates in the inhibition of nucleases in biological samples. A chelator can be, for example, ethylene glycol tetraacetic acid (EGTA), (2-hydroxyethyl)ethylenediaminetriacetic acid (HEDTA), diethylene triamine pentaacetic acid (DTPA), nitrilotriacetic acid (NTA), ethylenediaminetriacetic acid (EDTA), 1,2-cyclohexanediaminetetraacetic acid (CDTA), N,N-bis(carboxymethyl)glycine, triethylenetetraamine (TETA), tetraazacyclododecanetetraacetic acid (DOTA), desferioximine, citrate anhydrous, sodium citrate, calcium citrate, ammonium citrate, ammonium bicitrate, citric acid, diammonium citrate, ferric ammonium citrate, and lithium citrate. These chelating agents may be used singly or in combination of two or more thereof.

In one embodiment, there is provided a method of stabilizing nucleic acid contained in a biological sample at ambient temperature comprising the steps of: a) obtaining a biological sample; b) contacting the biological sample with an aqueous composition to form a mixture, wherein the aqueous composition comprises: (i) a denaturing agent selected from sodium dodecyl sulphate (SDS), lithium dodecyl sulphate, or a guanidinium salt; (ii) aurintricarboxylic acid (ATA), or a salt thereof; and (iii) at least one of a chelating agent and a buffering agent; wherein the composition has a pH of 4.9 or less; c) homogenizing the mixture of (b) to form a homogeneous mixture; and d) storing the homogeneous mixture at ambient temperature.

In another embodiment, there is provided an aqueous composition for stabilizing nucleic acids contained in a biological sample at ambient temperature, comprising: (i) a denaturing agent selected from sodium dodecyl sulphate (SDS), lithium dodecyl sulphate, or a guanidinium salt; (ii) aurintricarboxylic acid (ATA), or a salt thereof; and (iii) at least one of a chelating agent and a buffering agent; wherein the composition has a pH of 4.9 or less.

In another embodiment of the method and composition of the present application, the aqueous composition comprises (i) a denaturing agent selected from sodium dodecyl sulphate (SDS) or lithium dodecyl sulphate; (ii) aurintricarboxylic acid (ATA), or a salt thereof; and (iii) a chelating agent and, optionally, a buffering agent; wherein the composition has a pH of 4.9 or less. In one embodiment, the denaturing agent is lithium dodecyl sulphate or SDS and is present at a concentration of from about 2% to about 12% (w/v), or from about 3% to about 9% (w/v), or from about 4% to about 8% (w/v), or about 4% (w/v), or about 8% (w/V).

In another embodiment of the method and composition of the present application, the aqueous composition comprises (i) a denaturing agent selected from a guanidinium salt; (ii) aurintricarboxylic acid (ATA), or a salt thereof; and (iii) a buffering agent; wherein the composition has a pH of 4.9 or less. In one embodiment, the guanidinium salt is guanidinium thiocyanate or guanidinium hydrochloride. In another embodiment, the guanidinium salt is guanidinium thiocyanate. In yet another embodiment, the guanidinium thiocyanate is present at a concentration of from about 1 M to about 6 M, or from about 1 M to about 4 M, or from about 1.5 M to about 2.5 M, or about 2 M. In another embodiment, the guanidinium salt is guanidinium hydrochloride. In still another embodiment, the guanidinium hydrochloride is present at a concentration of from about 1 M to about 6 M, or from about 2 M to about 5 M, or from about 3.5 M to about 4.5 M, or about 4 M.

As the skilled worker will appreciate, specific embodiments described herein with respect to the aqueous composition for stabilizing nucleic acids contained in a biological sample at ambient temperature are also applicable to the related methods described herein.

In general, the pH of the present aqueous composition can be maintained in the desired range using one or more appropriate buffering agents. In accordance with one embodiment, the composition comprises one, two, or more buffering agents (non-limiting examples being acetate buffer and citrate buffer, such as sodium acetate, potassium acetate, ammonium acetate, sodium citrate, and ammonium citrate) with pKₐ values, logarithmic acid dissociation constants, at 25° C. ranging from 3 to 6.5 to maintain a pH of 4.9 or less. In one embodiment, the buffering agent is sodium acetate. It is noted that PAAc, ATA and CDTA can also contribute to the buffering capacity of the present composition, when present.

An acid dissociation constant, Kₐ, is a quantitative measure of the strength of an acid in solution. The larger the Kₐ value, the more dissociation of the molecules in solution and thus the stronger the acid. Due to the many orders of magnitude spanned by Kₐ values, a logarithmic measure of the acid dissociation constant, pKₐ, is more commonly used in practice. The larger the value of pKₐ, the smaller the extent of dissociation at any given pH, i.e., the weaker the acid. In living organisms, acid-base homeostasis and enzyme kinetics are dependent on the pKₐ values of many acids and bases present in the cell and in the body. In chemistry, knowledge of pKₐ values is necessary for the preparation of buffer solutions and is also a prerequisite for a quantitative understanding of the interaction between acids or bases and metal ions to form complexes. One skilled in the art will understand that a given compound/buffer can buffer the pH of a solution only when its concentration is sufficient and when the pH of the solution is close (within about one pH unit) to its pKₐ. In one embodiment, the pH of the present composition is 4.9 or less. In another embodiment, the pH of the composition is from 3.8 to 4.9, or from 4.3 to 4.7. The amount of buffering agent(s) in the aqueous composition can be from about 10 mM to about 500 mM, or from about 25 mM to about 250 mM, or from about 25 mM to about 150 mM, or from about 25 mM to about 75 mM, or about 50 mM, for example.

In another embodiment, the chelating agent in the aqueous composition is selected from, for example, ethylene glycol tetraacetic acid (EGTA), (2-hydroxyethyl)ethylenediaminetriacetic acid (HEDTA), diethylene triamine pentaacetic acid (DTPA), nitrilotriacetic acid (NTA), ethylenediaminetriacetic acid (EDTA), 1,2-cyclohexanediaminetetraacetic acid (CDTA), N,N-bis(carboxymethyl)glycine, triethylenetetraamine (TETA), tetraazacyclododecanetetraacetic acid (DOTA), desferioximine, citrate anhydrous, sodium citrate, calcium citrate, ammonium citrate, ammonium bicitrate, citric acid, diammonium citrate, ferric ammonium citrate, lithium citrate, or a combination thereof. In another embodiment, the chelating agent is selected from CDTA, DTPA, DOTA, TETA, desferioximine, or chelator analogs thereof. In another embodiment, the chelating agent is CDTA. In another embodiment, the chelating agent is present in the aqueous composition in an amount of from about 25 mM to about 250 mM, or from about 50 mM to about 150 mM, or about 100 mM.

In another embodiment, the aqueous composition comprises a salt, which is preferably an inorganic salt, such as ammonium sulphate, or a lithium or sodium salt, that is soluble in the aqueous composition. In one embodiment, the salt is lithium sulphate (Li₂SO₄), lithium chloride (LiCl), sodium chloride (NaCl), or any combination thereof. In another embodiment, the inorganic salt is present at a concentration of from about 100 mm to about 750 mM, or from about 200 mM to about 600 mM, or about 500 mM, or about 250 mM.

In another embodiment, the ATA, or the salt thereof, is present in the aqueous composition at a concentration of from about 2.5 mM to about 50 mM, or from about 5 mM to about 15 mM, or about 10 mM. Salts of ATA can include ammonium salts, sodium salts, and the like.

In another embodiment, the aqueous composition further comprises polyacrylic acid (PAAc), or a salt thereof. In one embodiment, the PAAc, or the salt thereof, has a molecular weight of from about 2,000 to about 10,000, or from about 2,000 to about 5,000, or about 5,000. In another embodiment, the PAAc, or the salt thereof, is present at a concentration of from about 5 mg/mL to about 20 mg/mL, or from about 5 mg/mL to about 15 mg/mL, or about 10 mg/mL. Salts of PAAc can include ammonium salts, sodium salts, and the like.

In one embodiment of the above-noted method and composition, the ambient temperature is from about 15° C. to about 25° C. In another embodiment, ambient temperature is −20° C. or 37° C. or 50° C., to simulate conditions encountered in the field. In another embodiment, the biological sample is a saliva sample or a fecal sample. In another embodiment, the biological sample is a saliva sample obtained from a mammal, such as a human. In another embodiment, the biological sample is a feces sample obtained from a mammal, such as a human.

In another embodiment, the biological sample is saliva and the saliva sample is collected using a device such as, for example, those described in WO2007/068094 entitled “CONTAINER SYSTEM FOR RELEASABLY STORING A SUBSTANCE”, WO2010/020043 entitled “SAMPLE RECEIVING DEVICE”, and WO2010/130055 entitled “CLOSURE, CONTAINING APPARATUS, AND METHOD OF USING SAME”.

In another embodiment, the biological sample is a fecal sample, and the fecal sample is collected using a device such as that described in WO2015172250 entitled “DEVICE FOR COLLECTING, TRANSPORTING AND STORING BIOMOLECULES FROM A BIOLOGICAL SAMPLE”.

In still another embodiment, the biological sample can be collected in a standard, commercially-available laboratory or transport tube (e.g. 10 mL round-bottom tube (92×15.3 mm), Cat. No. 60.610; Sarstedt, or larger tube depending on the sample type and size). The tube containing the biological sample and aqueous composition can be sealed with an appropriate cap, and the combined sample and aqueous composition can be gently mixed, for example by inverting the tube.

The biological sample should preferably be mixed immediately with the aqueous composition at the point of collection. Otherwise, samples should be stored and/or transported on ice packs or refrigerated before mixing with the composition.

As the skilled worker will appreciate, the aqueous composition (“chemistry”) described herein can be combined with the biological sample in a variety of ratios. Samples can be mixed with the chemistry at a ratio of 1:1 to 1:12 (vol/vol depending on the sample type).

In another embodiment of the method and composition of the present application, the nucleic acid contained in the biological sample is deoxyribonucleic acid (DNA). In another embodiment of the method and composition of the present application, the nucleic acid contained in the biological sample is ribonucleic acid (RNA). In yet another embodiment, the method and composition of the present application stabilize both DNA and RNA contained in a biological sample. In another embodiment, the method renders the nucleic acid stable for at least 7 days at a temperature of from about 15° C. to about 25° C., or for at least 14 days at a temperature of from about 15° C. to about 25° C.

Methods of assessing stabilization of nucleic acids are known to the skilled worker and/or are outlined in further detail in the Materials and Methods section and Examples described below. For example, stabilization of DNA can be determined by the ability to recover high molecular weight DNA (>8 kb in size) from the samples. DNA was recovered from biological samples using a commercial kit that relies on mechanical lysis (bead beating) for lysis as this approach enables recovery of DNA from both gram-positive and gram-negative bacteria. DNA was purified on silica columns. Stabilization of RNA can be determined by minimal loss of rRNA doublet integrity over time as compared to samples extracted at baseline. Total endogenous RNA was recovered from samples using commercial kits as described above for DNA. Exogenous RNA was purified with silica columns. Well-stabilized RNA samples will have both 16S and 23S rRNA bands clearly visible and preferably will have minimal visible smearing (which is evidence of RNA degradation by-products), compared to samples extracted at baseline. Microbial DNA & RNA extraction procedures involve direct cell lysis that can be chemical, mechanical and enzymatic, followed by removal of cell fragments and nucleic acid precipitation and purification. Additional enzyme inhibitor (for example humic acids, polyphenols, polysaccharides and heme) removal step prior to nucleic acid precipitation can be achieved by precipitation and centrifugation, cesium chloride density gradient ultracentrifugation, chromatography, electrophoresis or dialysis and filtration; its need is dependent on the sample type being processed. Samples exhibiting stabilization of nucleic acids will appear similar to those obtained at T0 and/or will exhibit sharper/clearer DNA/rRNA bands relative to control samples (wherein the control samples lack one or more components/properties of the test composition).

In still yet another embodiment, the aqueous composition comprises, consists essentially of, or consists of: (i) a denaturing agent selected from lithium dodecyl sulphate, SDS, or a combination thereof; (ii) aurintricarboxylic acid (ATA), or a salt thereof; (iii) a chelating agent; (iv) polyacrylic acid (PAAc), or a salt thereof; and (v) an inorganic salt, wherein the inorganic salt is a lithium salt or a sodium salt that is soluble in the aqueous composition. In still yet another embodiment, the aqueous composition comprises, consists essentially of, or consists of: (i) SDS; (ii) aurintricarboxylic acid (ATA), or a salt thereof; (iii) CDTA; (iv) polyacrylic acid (PAAc), or a salt thereof, having a molecular weight of from about 2,000 to about 10,000, or from about 2,000 to about 5,000, or about 5,000; and (v) lithium sulphate, lithium chloride, sodium chloride, or any combination thereof. In yet another embodiment, the SDS is present at a concentration of from about 2% to about 12% (w/v), or from about 3% to about 9% (w/v), or from about 4% to about 8% (w/v), or about 4% (w/v), or about 8% (w/v); the ATA, or the salt thereof, is present at a concentration of from about 2.5 mM to about 50 mM, or from about 5 mM to about 15 mM, or about 10 mM; the chelating agent is present at a concentration of from about 25 mM to about 250 mM, or from about 50 mM to about 150 mM, or about 100 mM; the PAAc, or the salt thereof, is present at a concentration of from about 5 mg/mL to about 20 mg/mL, or from about 5 mg/mL to about 15 mg/mL, or about 10 mg/mL; and the inorganic salt is present at a concentration of from about 100 mM to about 750 mM, or from about 200 mM to about 600 mM, or about 500 mM, or about 250 mM.

In still another embodiment, there is provided a stabilized biological composition comprising the above-noted aqueous composition in combination with a biological sample. In another embodiment, the biological sample is a saliva sample or a fecal sample, optionally wherein the biological sample is obtained from a mammal, such as a human.

### EXAMPLES

### Materials & Methods

### Sample Collection

Human saliva and stool samples were collected under DNA Genotek's IRB protocol. Specifically, fresh raw saliva was collected in sterile tubes and kept on ice for a maximum of 2-3 hours until further processing. Saliva was mixed at 1:1 ratio with formulations to be tested and aliquoted for total nucleic acid extraction, nuclease assay (RNAseAlert®) or Quickscreen assay (see below). Stool samples were collected directly into OMNIgene®⋅GUT OMR-200 Kits (DNA Genotek Inc., Canada) filled with the formulations of interest. 0.1 to 0.2% antifoam A concentrate (Sigma Aldrich, Cat #A5633-25G) was added directly to each stool collection tube to avoid excessive foaming during sample homogenization. Samples were returned to the laboratory within a few hours of collection and aliquoted for further processing (Nuclease assay, Quickscreen assay and/or total nucleic acid extractions).

### Quickscreen Assay

The Quickscreen assay (QS) was developed as a means to assess nuclease release and activity in samples collected in lytic formulations of the present application. Briefly, saliva and stool samples from numerous donors were mixed with the formulations to be tested and incubated for 30 minutes to 2 hours at room temperature (allowing for chemistry-driven lysis). For saliva samples, the mixture was directly spiked with purified total RNA from Francisella philomiragia at a final RNA concentration of 30-40 ng/μL. For stool samples, the fecal matrix was removed by centrifugation prior to spiking Francisella philomiragia total RNA at 30-40 ng/μL. RNA was then purified at baseline (T0) and after 2-3 days of storage at room temperature, using Qiagen's RNeasy® MinElute® Cleanup Kit (Catalogue No. 74204). RNA stability and chemistry performance were assessed by comparing RNA processed at various time points versus T0 (baseline).

### Nuclease Assay (RNAseAlert®)

The RNaseAlert® assay (IDT, Cat #11-04-02-03) was also used as an alternative to the Quickscreen assay to quantify RNAse activity in collected samples. Briefly, saliva and stool samples were mixed with the formulations to be tested and incubated for 30 minutes to 2 hours at room temperature. For saliva samples, a 45 μL aliquot was then directly transferred to a fresh tube and 5 μl (10 pmoles) of the RNAseAlert® substrate was added. Samples were incubated at room temperature for 1-3 h in presence of the substrate and diluted 1:10 to 1:25 in 0.1 M Tris pH 8.0 before reading fluorescence (Ex=490 nm, Em=520 nm) on a Tecan Infinite M200 microplate reader. For stool samples, the fecal matrix was pelleted by centrifugation and the pre-cleared 45 μL aliquot was then transferred to a fresh tube and mixed with 5 μL of RNAseAlert® substrate. Fluorescence was measured as described above for saliva samples.

### Total Nucleic Extractions

For saliva and stool sample total nucleic acid extractions, a 200 μL aliquot was taken and extracted using Qiagen's RNeasy® PowerMicrobiome Kit (Catalogue No. 26000-50), according to the manufacturer's instructions. Bead beating was performed in the presence of phenol-chloroform-isoamyl alcohol (Sigma-Aldrich, Catalogue No. 77617) and 2-mercaptoethanol (Sigma-Aldrich, Catalogue No. M6250) and on-column DNAse treatment was skipped in order to isolate both DNA and RNA. The final eluate was split into two fractions and treated with either DNAse I (Lucigen, Catalogue No. DB0715K) or RNAse A (Thermo Fisher Scientific, Catalogue No. EN0531). DNA was run on genomic DNA screentapes (Agilent, Catalogue No. 5067-5365), while RNA samples were cleaned-up with Qiagen's RNeasy® MinElute® Cleanup Kit (Catalogue No. 74204), and then run on RNA Screentapes (Catalogue No. 5067-5576) on the Agilent TapeStation 4200 system. Chemistry performance was assessed by comparing DNA/RNA quality at various time points verses baseline (T0).

For stool samples stabilized in guanidinium based chemistries, a 200 μL aliquot was taken and RNA and DNA was extracted in two separate fractions, using ZymoBIOMICS DNA/RNA Miniprep Kit (Cat #. R2002), following manufacturer's instructions. RNA was run on RNA Screentapes as described above.

For RT-qPCR testing, total nucleic acids were extracted from a 200 μl saliva aliquot using the MagMax™ viral pathogen nucleic acid extraction kit (Catalogue No. A48310), according to the manufacturer's instructions.

### Isolation of the Pellet and Supernatant Fractions

To isolate fecal pellets and supernatants, a 200 μL aliquot of the sample collected in formulations of interest was transferred to a 1.5 ml tube at the indicated time point, and centrifuged at 8000×g for 5 minutes in order to pellet intact cells (as well as the fecal matrix). The supernatant was transferred to a fresh tube and total nucleic acids were extracted using Qiagen's RNeasy® PowerMicrobiome Kit (the bead beating step was skipped). Total nucleic acids were also extracted from the pellet fraction using Qiagen's RNeasy® PowerMicrobiome Kit (bead beating step was included for pellet extractions). DNA and RNA extracted from the pellet and supernantant were then processed as described above.

### Metatranscriptomics Sequencing and Analysis

In RNA sequencing experiments, rRNA was depleted from purified total RNA samples using Illumina's RiboZero plus kit (Cat #20037135). Depleted mRNA was then prepped using Illumina's stranded total RNA Prep kit (Cat #20040529) as per manufacturer's recommendations. Final libraries were quantified with the Quant-iT™ PicoGreen™ dsDNA Assay Kit (Cat #P7589), pooled and then sequenced using a 75 cycles NextSeq 500/550 High Output Kit v2.5 (Cat #20024906).

Demultiplexed, FASTQ files were trimmed and quality filtered using in-house scripts. Briefly, reads were filtered and trimmed using kneaddata v0.6.1 with Bowtie2 v.2.3.4.1 [1] to remove contaminating human and ribosomal rRNA genes by mapping against hg37 and SILVA v128 references respectively. Timmomatic v0.38 was used to trim low quality bases (<Q20) and remove any leftover sequence adapters. The resultant trimmed and filtered reads were mapped using Kaiju v1.6.3 [2] to the bacterial proGenomes database v2 for taxonomic assignment.

Using R [3], the mapped read counts table was filtered to keep reads assigned to taxonomic bins occurring in at least 2 samples and having a total of at least 10 mapped reads. Total read counts per sample were then aggregated to different taxonomic levels of annotation (Species, Genus, Family, Order, Phylum), and percent abundance was calculated as (reads/taxonomic bin)/(total reads per sample). For visual presentation, only the top 10 most abundant taxonomic groups are shown, with the remaining reads grouped into “Other”.
[1] Langmead, B., & Salzberg, S. L. (2012). Fast gapped-read alignment with Bowtie 2. Nature methods, 9(4), 357.
[2] Menzel, P., Ng, K. L., & Krogh, A. (2016). Fast and sensitive taxonomic classification for metagenomics with Kaiju. Nature communications, 7(1), 1-9.R Core Team (2018).
[3] R: A language and environment for statistical computing. R Foundation for Statistical Computing, Vienna, Austria. URL https://www.R-project.org/.

### RT-qPCR

For human and viral RNA stability testing, a 5 μL total nucleic acid aliquot extracted using MagMax™ viral pathogen nucleic acid extraction kit was used as template in a 1-step RT-qPCR reaction using the GoTaq® Probe RT-qPCR from Promega (Catalogue No. A6120), following the manufacturer's instructions. Human Histatin 3 mRNA levels were measured using a Thermo Fischer Scientific Taqman assay id Hs00264790_m1 (HTN3) (Catalogue No. 4331182). Primers and probes targeting the matrix gene of influenza A¹ and nucleocapsid gene of RSV A² were used to assess viral RNA stability.

Primers for Influenza matrix gene were as follows (based on WHO guidelines¹): Forward primer 5′-CCGAGGTCGAAACGTACGTTCTCTCTATC-3′ (SEQ ID NO: 1); Reverse primer 5′-TGACAGGATTGGTCTTGTCTTTAGCCATTCCA-3′ (SEQ ID NO: 2); Probe 5′-ATCTCGGCTTTGAGGGGGCCTG-3′ (SEQ ID NO: 3).

The RSV A primers used in the experiments are known in the art² and are as follows: Forward primer 5′-TGCTAAGACTCCCCACCGTAAC-3′ (SEQ ID NO: 4); Reverse primer 5′-GGATTTTTGCAGGATTGTTTATGA-3′ (SEQ ID NO: 5); Probe 5′-CACTTGCCCTGCACCA-3′ (SEQ ID NO: 6).

References for RT-qPCR Materials and Methods:
¹WHO information for the molecular detection of influenza viruses. July 2017. Accessed from: https://www.who.int/influenza/gisrs_laboratory/WHO_information_for_the_molecular_detection_of_influenza_viruses_20171023_Final.pdf.
²Essaidi-Laziosi M, Lyon M, Mamin A, Fernandes Rocha M, Kaiser L, Tapparel C. A new real-time RT-qPCR assay for the detection, subtyping and quantification of human respiratory syncytial viruses positive- and negative-sense RNAs. J Virol Methods. 2016 Sep;235:9-14. doi: 10.1016/j.jviromet.2016.05.004. Epub 2016 May 11. PMID: 27180039.

### Next Generation Sequencing

16S amplicon sequencing (V3-V4 region) was performed following Illumina's standard 16S library preparation guidelines. Prior to library preparation, RNA samples were reverse transcribed using M-MLV reverse transcriptase (Invitrogen, Cat No. 28025013) following the manufacturer's protocol using 100 ng total RNA as input. Paired-end reads were generated on Illumina's MiSeq system with the 600 cycles reagent kit (Catalogue No. MS-102-3003).

For metatranscriptomic analysis of stool RNA samples, 250 ng total RNA input was added to RiboZero Plus rRNA depletion reactions (Catalogue No. 20037135), supplemented with a custom microbiome depletion pool (DPM). Library Prep was then performed using the Total RNAPrep kit (Catalogue No. 20040529). Libraries were sequenced on Illumina's NextSeq system using a 2×150 bp high-output kit (Catalogue No. 20024908).

### Example 1. Effect of Nuclease Inhibitors on Nucleic Acid Stability in Biological Samples

Inhibition or inactivation of nucleases is critical to maintain the integrity of nucleic acids in complex biological samples. Numerous inhibitors and reducing agents were tested for their ability to prevent nuclease activity in saliva samples obtained from nuclease-rich donors. The saliva samples were mixed with 4% SDS/100 mM CDTA/500 mM Li₂SO₄, pH 5.2 using the Quickscreen assay (see Materials & Methods). Inhibitors tested included pontacyl violet 6R (5-10 mM; Santa Cruz Biotechnology, Catalogue No. sc-489792), β-2-mercaptoethanol (1% vol/vol; Sigma-Aldrich, Catalogue No. M6250), dithiothreitol (DTT) (20 mM; Invitrogen, Catalogue No. P2325), tris(2-carboxyethyl)phosphine (TCEP) (20 mM; Sigma-Aldrich, Catalogue No. C4706), ribonucleoside vanadyl complexes (RVC) (2-10 mM; Sigma-Aldrich, Catalogue No. 94740), 5,5-dithio-bis-(2-nitrobenzoic acid) (DTNB) (5-20 mM; Sigma-Aldrich, Catalogue No. D8130), glucosamine 6-phosphate (10 mM; Sigma-Aldrich, Catalogue No. G5509), 7-nitroindole-2-carboxylic acid (10 mM; Fisher Scientific, Catalogue No. AAB2078603), sodium p-toluenesulfonate (5 mM; Sigma-Aldrich, Catalogue No. 152536), epigallocatechin gallate (EGCG) (5 mM; Sigma-Aldrich Catalogue No. E4143), aurintricarboxylic acid (ATA) (2.5-50 mM; Sigma-Aldrich, Catalogue No. A1895), as well as commercially available inhibitors such as recombinant human placental RNAse inhibitor (4 units/μL; BLIRT, Catalogue No. RT35-020). Degradation of RNA in the saliva samples obtained from nuclease-rich donors was observed after a 2 day incubation at room temperature under the experimental conditions in the presence of all of the above-noted inhibitors/reducing agents, except for ATA. ATA surprisingly out-performed all inhibitors/reducing agents tested in preventing degradation of RNA in saliva samples obtained from nuclease-rich donors. Unexpectedly, EGCG, which has a chemical structure similar to ATA, did not prevent degradation of RNA in saliva samples obtained from the same donors. Further, some of the inhibitors/reducing agents tested unexpectedly increased RNA degradation in specific samples, suggesting that they can promote RNAse activity in select samples.

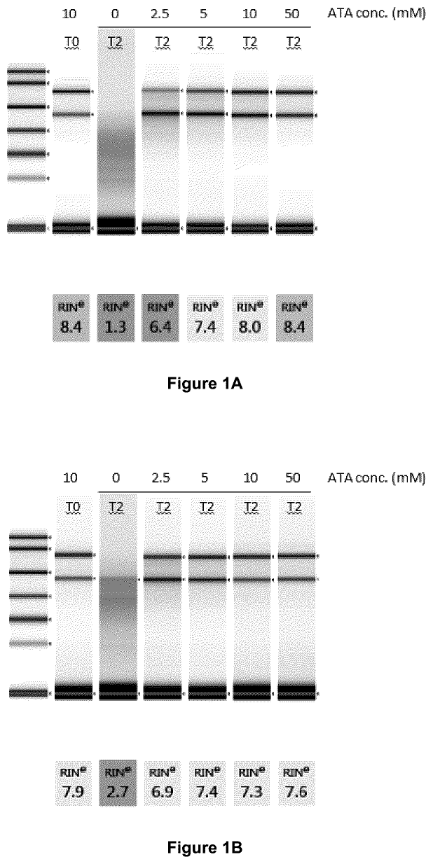

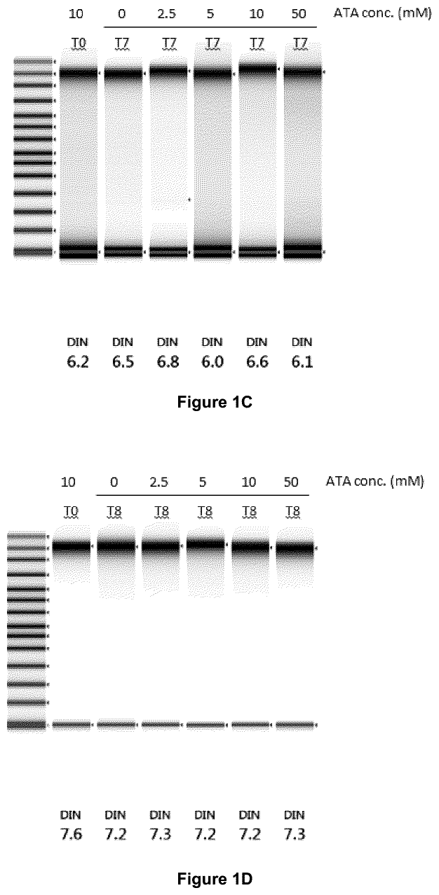

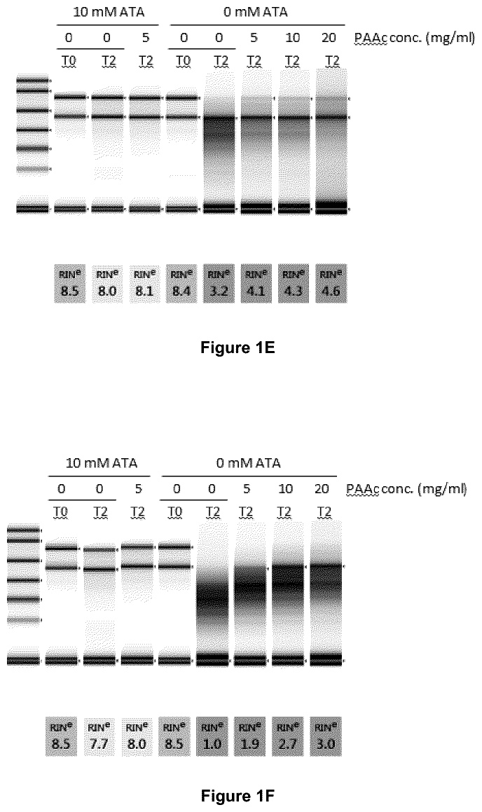

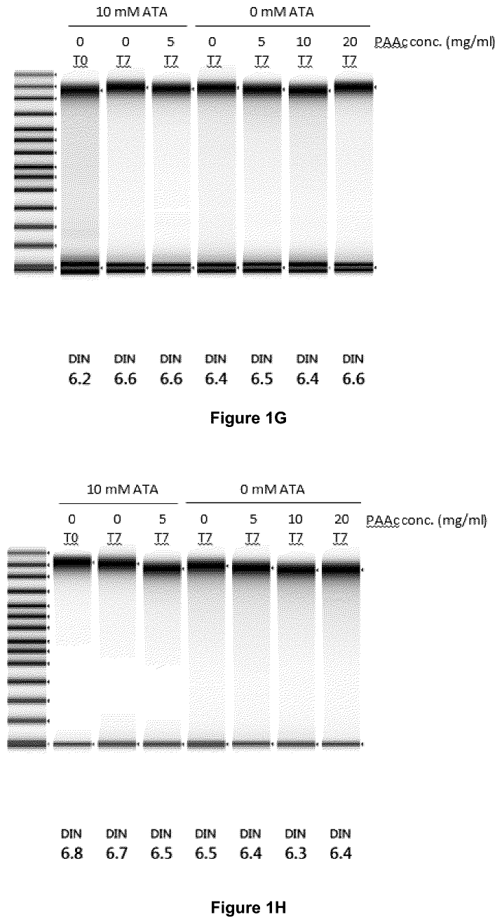

Saliva aliquots from two donors were mixed 1:1 with the present composition (4% SDS/100 mM CDTA/500 mM Li₂SO₄, pH 4.6), including increasing concentrations of ATA (0-50 mM). The aliquots were spiked with purified bacterial RNA (see Materials & Methods) and then stored at room temperature for two days. At T0 and T2, RNA was purified from each donor's aliquots using Qiagen's RNeasy® MinElute® Cleanup Kit and then visualized on the TapeStation 4200 system. In the absence of ATA, the spiked ribosomal RNA doublet was largely degraded and the RIN was 1.3-2.7. As the concentration of ATA increased to 50 mM, the ribosomal RNA doublet became increasingly defined and the RINs increased incrementally to 7.6-8.4 (see FIG. 1A-B) for both donors, demonstrating robust inhibition of RNAses by ATA.

Endogenous genomic DNA was also purified from each donor's saliva sample using Qiagen's RNeasy® PowerMicrobiome Kit at T0 and T7. The final total nucleic acid eluate was treated with RNAse A, and DNA was run on genomic DNA screentapes on Agilent's TapeStation 4200 System. Unlike RNA, genomic DNA from both donors remained intact in the present composition with or without ATA (see FIGS. 1C-D).

In addition to small molecule inhibitors, various polyanionic compounds were evaluated for their ability to inhibit RNAses in biological samples. Polyanionic compounds can bind and sequester proteins that are attracted to negative charges (such as nucleases). Specifically, poly-acrylic acid (PAAc) (5-20 mg/ml; Sigma-Aldrich, Catalogue No. 192031), heparin (Hep) (5 mg/ml; Sigma-Aldrich, Catalogue No. H3149), dextran sulfate (DS) (5-30 mg/mL; Sigma-Aldrich, Catalogue No. 51227), polyglutamic acid (PGA) (5 mg/ml; Sigma-Aldrich, Catalogue No. P4636), chitosan (0.1% vol/vol; Sigma-Aldrich, Catalogue No. 448869) and polyvinylsulfonic acid (PVSA) (10 mg/mL; Sigma-Aldrich, Catalogue No. 278424) were tested with saliva samples collected from nuclease-rich donors in 4% SDS/100 mM CDTA/500 mM Li₂SO₄, pH 5.2 using the Quickscreen assay. Degradation of RNA in the saliva samples obtained from nuclease-rich donors was observed after a 2 day incubation at room temperature under the experimental conditions in the presence of all of the above-noted polyanionic compounds, except for PAAc. PAAc surprisingly out-performed all polyanionic compounds tested in preventing degradation of RNA in saliva samples obtained from nuclease-rich donors. This suggests that PAAc, unlike the other polyanionic compounds tested, is able to effectively bind the broad range of nucleases found in complex biological samples.

Saliva aliquots from two donors were mixed 1:1 with the present composition (4% SDS/100 mM CDTA/500 mM Li₂SO₄, pH 4.8), including 10 mM ATA and/or increasing concentrations of PAAc (0-20 mg/mL). The aliquots were spiked with purified bacterial RNA (see Materials & Methods) and then stored at room temperature for two days. At T0 and T2, spiked RNA was purified from each donor's aliquots using Qiagen's RNeasy® MinElute® Cleanup Kit and then visualized on Agilent's TapeStation 4200 system. In the presence of ATA, the ribosomal RNA doublet was intact and the RIN was high (7.7-8.0). In the presence of increasing concentrations of PAAc although the quality of the RNA improved, the ribosomal RNA doublet was not intact; RIN values increased incrementally to 3.0-4.6 (see FIGS. 1E-F). There was no significant improvement in the RIN when samples were collected in a composition containing both ATA and PAAc (FIGS. 1E-F).

Endogenous genomic DNA was also purified from each donor's sample using Qiagen's RNeasy® PowerMicrobiome Kit at T0 and T7. The total nucleic acid eluate was treated with RNAse A, and DNA was run on genomic DNA screentapes on Agilent's TapeStation 4200 System. Genomic DNA from both donors remained intact in the present composition regardless of the presence of PAAc or ATA (see FIGS. 1G-H).

### Example 2. Stability of Saliva and Stool Nucleic Acids is Surprisingly Dependent on Low pH of the Present Composition

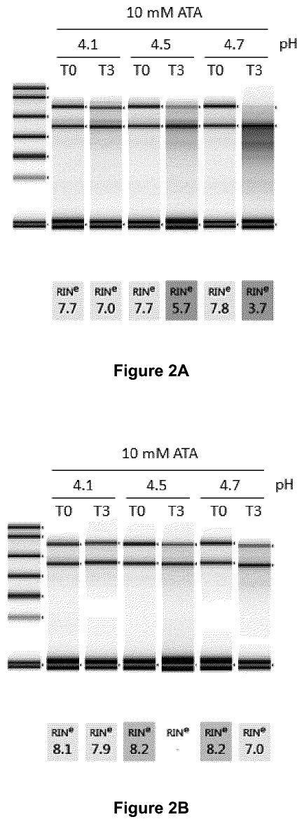

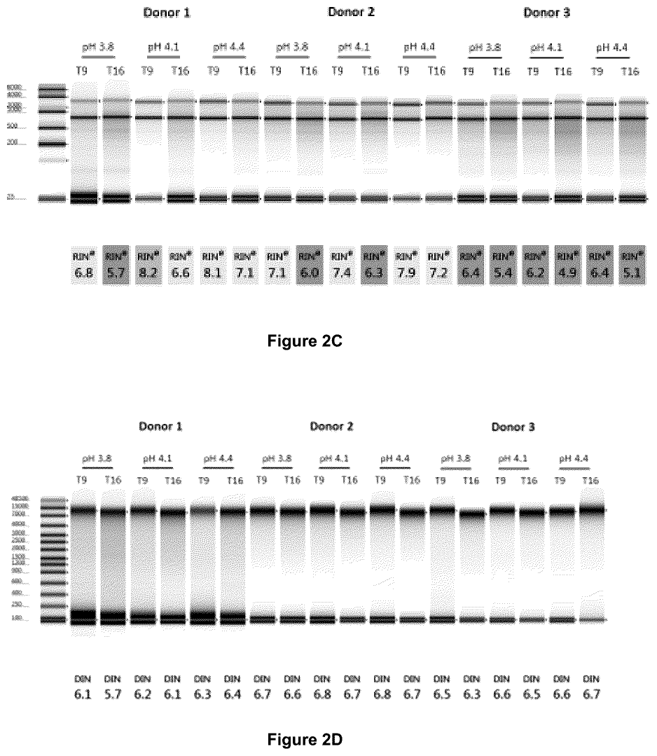

The present composition (4% SDS/100 mM CDTA/500 mM Li₂SO₄/10 mM ATA) was prepared and the final pH was adjusted to 4.1, 4.5, and 4.7. Two healthy donors provided a saliva sample and aliquots were mixed 1:1 with the present compositions, spiked with purified bacterial RNA for Quickscreen analysis (see Materials & Methods), and stored at room temperature for up to 3 days. After approximately two hours (T0) and 3 days (T3), the RNA spike-in was purified from each donor's aliquots using Qiagen's RNeasy® MinElute® Cleanup Kit and then visualized on the TapeStation 4200 system (Agilent) (see FIG. 2A-B).

For both donors, FIG. 2A and 2B demonstrate increased RNA stability when the pH of the composition decreases from pH 4.7 towards pH 4.1. At pH 4.1, the ribosomal RNA doublet is largely intact following three days incubation at room temperature. However, as the pH of the composition increases to 4.5 and 4.7, the ribosomal RNA bands show slight signs of degradation as shown by a decrease in RNA Integrity Number (RIN) and a slightly fainter upper band in the RNA doublet.

OMNIgene®⋅GUT kits (DNA Genotek Inc.) were filled with 4 mL of 4% SDS/100 mM CDTA/500 mM Li₂SO₄/10 mM ATA/10 mg/mL PAAc with the final pH adjusted to 3.8, 4.1, and 4.4. Three healthy donors dispensed approximately 400-500 mg of stool into each kit and returned the kits to the laboratory where they were stored at room temperature for up to 16 days. After nine days (T9) and 16 days (T16), endogenous total nucleic acids were purified from 200 μL aliquots using Qiagen's RNeasy® PowerMicrobiome Kit (see Materials & Methods). An aliquot of each total nucleic eluate was treated with DNase and RNA was then purified with the RNeasy® MinElute Clean up kit, and run on RNA screentapes on the TapeStation 4200 system (Agilent). Conversely, the remainder of the eluate was treated with RNAse A, and then DNA was run on genomic DNA screentapes (see FIGS. 2C-D).

Compositions with low pHs (3.8-4.4) maintained RNA stability over time in stool samples as visualized by intact RNA doublets. The RIN values increased slightly as the pH increased from 3.8 to 4.4 for two of the three donors (FIG. 2C). Surprisingly, for all three donors, high molecular weight genomic DNA (i.e. fragments >10 kb) was isolated after 9 and 16 days storage at room temperature despite the low pH (pH 3.8-4.4; FIG. 2D).

### Example 3. The Effect of Different Classes of Surfactants or Detergents on Nucleic Acid Stability in Biological Samples

In this example, the role of surfactants or detergents on nucleic acid stability in biological samples was examined. Cetyltrimethylammonium bromide (CTAB), a quaternary ammonium (cationic) surfactant, Tween 20, a polysorbate-type non-ionic surfactant, sodium lauroyl sarcosinate (SARK), an anionic surfactant, and sodium dodecyl sulfate (SDS), another example of an anionic surfactant were tested with both saliva and stool samples. Detergents (0-12% w/v) were added to a base composition comprised of 100 mM CDTA, 500 mM Li₂SO₄, and 10 mM ATA; pH of each mixture was adjusted to 4.71-4.73.

Within a few hours of collecting human saliva samples from three donors, aliquots were mixed 1:1 with the various compositions noted above. After a short incubation at room temperature, the aliquots were spiked with total RNA from Francisella philomiragia (Quickscreen, see Materials & Methods). Total RNA was purified at baseline (T0) and after 2 days at room temperature using RNeasy® MiniElute® Cleanup Kit (Qiagen) and then run on RNA Screentapes on the TapeStation 4200 system (Agilent). Total saliva nucleic acids were also extracted from an aliquot using the RNeasy® PowerMicrobiome Kit (Qiagen) and the final eluate was treated with RNAse A before running the DNA on genomic DNA screentapes (see Materials & Methods).

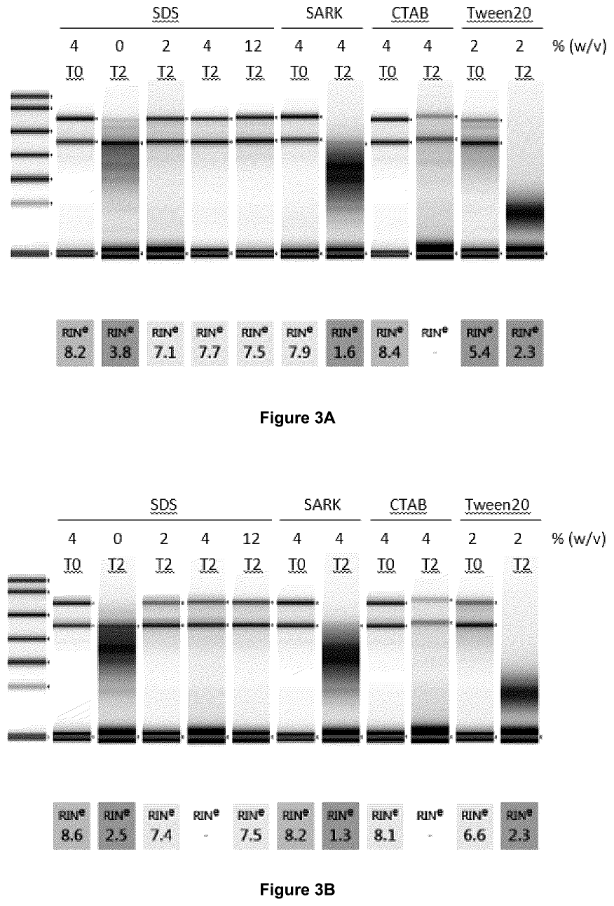

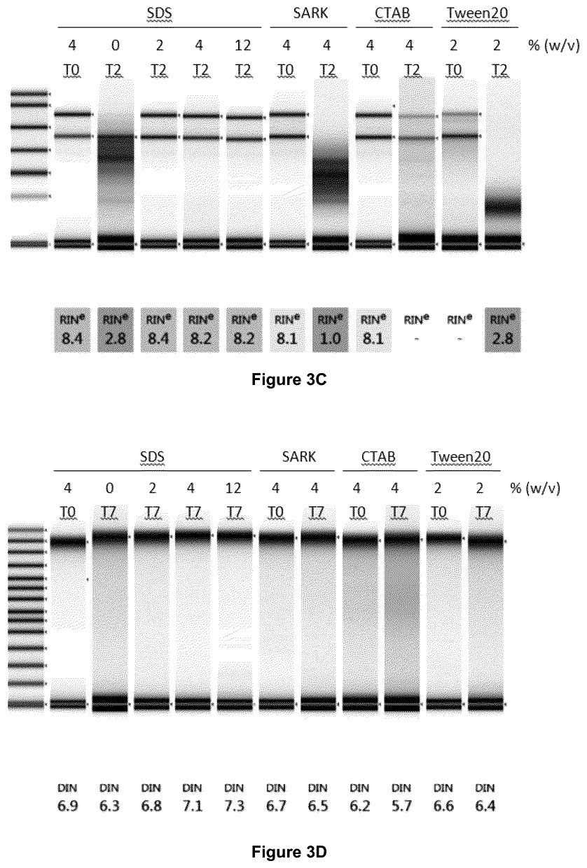

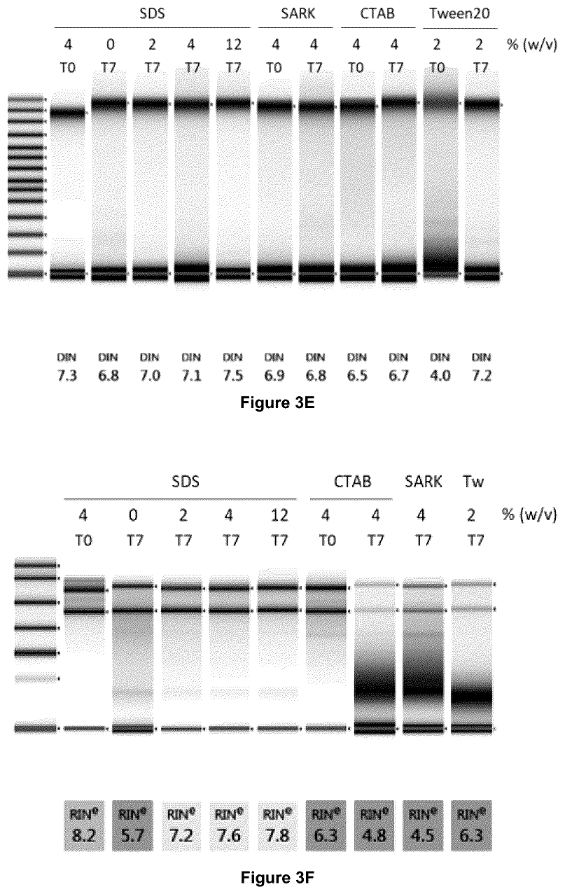

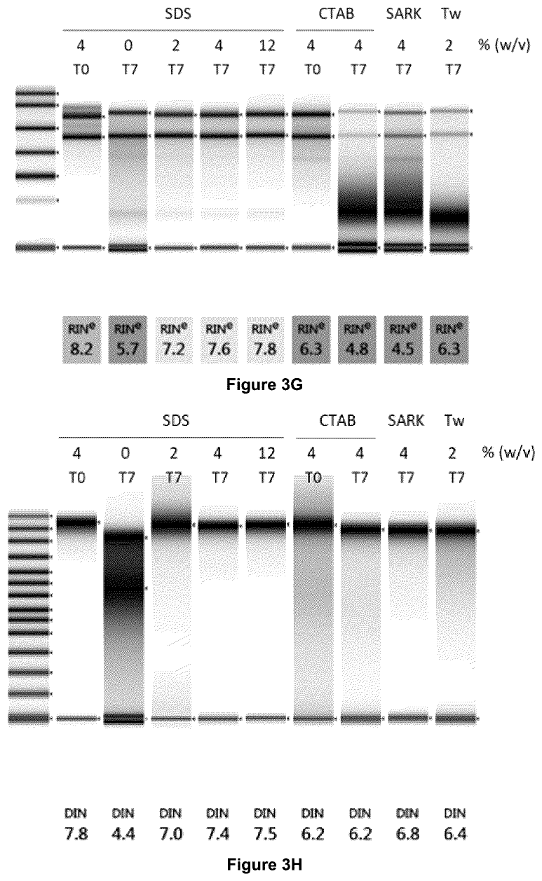

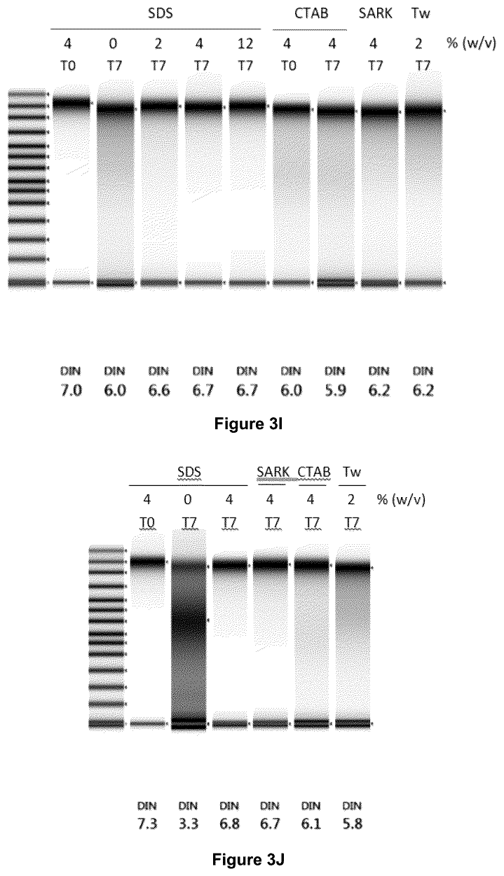

For all three saliva donors, degradation of RNA was observed in compositions lacking SDS. Specifically, the ribosomal RNA (rRNA) doublet was degraded and the RIN was significantly reduced for all 3 donors in compositions containing Sarkosyl, CTAB or Tween 20 (see FIG. 3A-C). Surprisingly, unlike Sarkosyl, SDS was able to preserve the integrity of rRNA in saliva samples from all three donors (see FIGS. 3A-C) stored at room temperature for 2 days. In contrast, genomic DNA was found to be high molecular weight under all conditions tested (FIG. 3D-E) for saliva from two donors.

For the collection of stool samples, OMNIgene®⋅GUT Kits (DNA Genotek Inc., Canada) were filled with 4 mL of the compositions defined above for saliva and distributed to two healthy donors. The donors dispensed approximately 400-500 mg of stool into each kit and returned the kits to the laboratory where they were stored at room temperature for up to 7 days. On the day of collection (T0) and after 7 days (T7), total nucleic acids were purified from 200 μL aliquots using Qiagen's RNeasy® PowerMicrobiome kit (see Materials & Methods). The final eluate was split into two fractions and treated with either DNase I or RNAse A. DNA was run on genomic DNA screentapes, while RNA samples were cleaned-up with RNeasy® MinElute R Cleanup Kit (Qiagen) and then run on RNA screentapes.

For both stool donors, degradation of RNA was observed in compositions lacking SDS. Specifically, the ribosomal RNA doublet was degraded and/or the RIN was significantly reduced in compositions containing Sarkosyl, CTAB or Tween 20 (see FIG. 3F-G). Surprisingly, unlike Sarkosyl, SDS was able to preserve the integrity of RNA in both stool samples (see FIGS. 3F-G) stored at room temperature for 7 days. In contrast, genomic DNA was found to be high molecular weight in stool samples collected in formulations containing SDS, CTAB, Sarkosyl or Tween 20 (FIG. 3H-J). Interestingly, the complete absence of a detergent resulted in loss of high molecular weight genomic DNA in stool samples in two of the three donors (FIG. 3H-J). Therefore, DNA stability in the absence of a detergent appears to be donor and sample specific.

### Example 4. The Effect of Different Chelating Agents on Nucleic Acid Stability in Complex Biological Samples

Example 2 demonstrates the importance of low pH for the stability of nucleic acids in biological samples. Chelating agents, in particular CDTA and EDTA, help contribute to the buffering capacity of the present composition in addition to their “traditional” role in chelation of divalent cations.

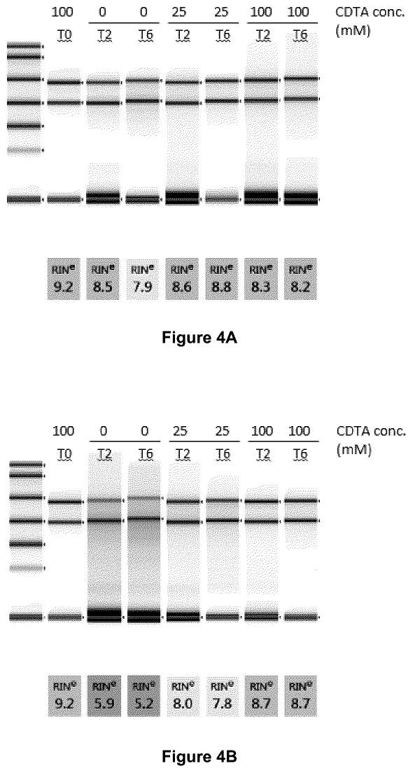

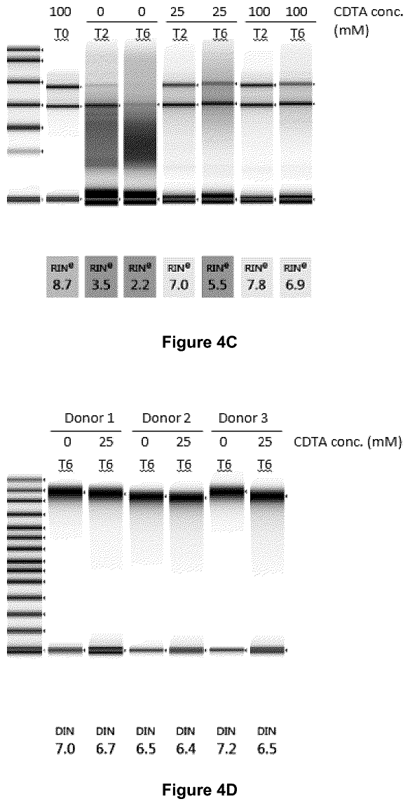

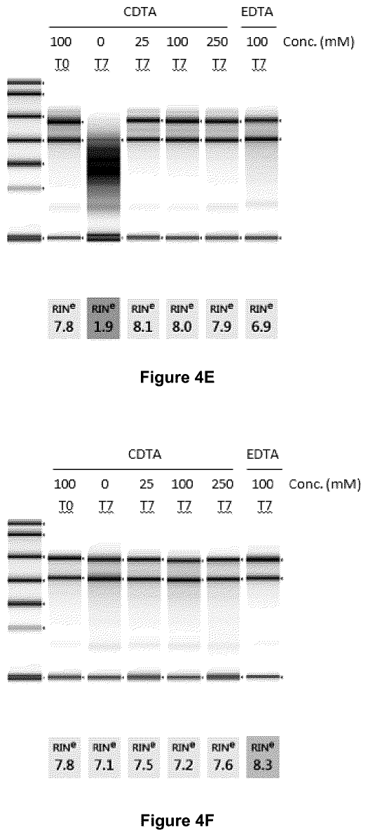

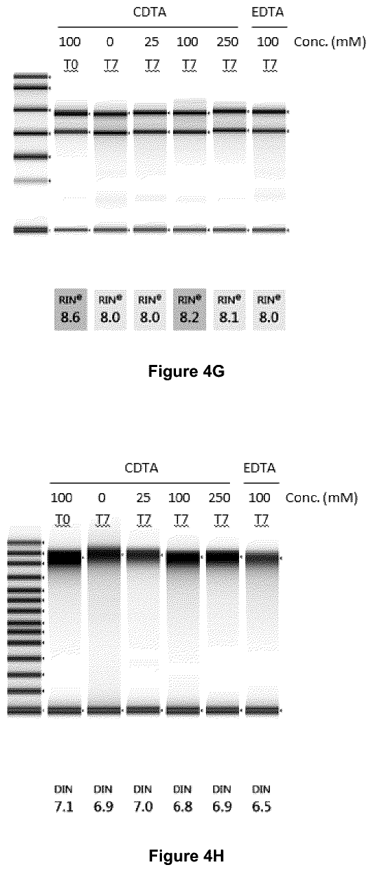

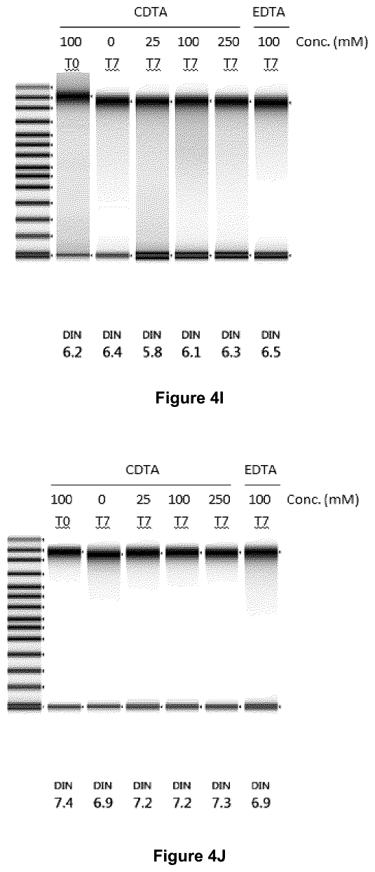

In order to test the efficacy of chelating agents in inhibiting nucleases found within complex biological samples, the present composition (4% SDS/500 mM Li₂SO₄/10 mM ATA/10 mg/mL PAAc) was prepared with increasing concentrations of CDTA (0-100 mM) and adjusted to pH 4.7. OMNIgene®⋅GUT Kits (DNA Genotek Inc., Canada) were filled with 4 mL of the compositions of interest and distributed to three healthy donors. The donors dispensed approximately 400-500 mg of stool into each kit and returned the kits to the laboratory where they were spiked with purified bacterial RNA for Quickscreen analysis (see Materials & Methods), and stored at room temperature for up to 6 days. After approximately two hours (T0), two days (T2) and six days (T6), RNA was purified from each donor's samples using Qiagen's RNeasy® MinElute® Cleanup Kit, ran on RNA screentapes and then visualized on the TapeStation 4200 system (Agilent) (see FIGS. 4A-C).

For all three donors, the absence of CDTA caused a reduction in RIN and/or degradation of the rRNA doublet after two and six days at room temperature. With the addition of 25 and 100 mM CDTA, RIN values increased and rRNA doublet remained intact (FIG. 4A-C) in all stool samples.

At T0 and after six days (T6), endogenous genomic DNA was purified from stool samples collected in the presence or absence of 25 mM CDTA using Qiagen's RNeasy® PowerMicrobiome Kit (see Materials & Methods). Total nucleic acids were treated with RNAse A, and then run on genomic DNA screentapes on the TapeStation 4200 system (Agilent). Interestingly, high molecular weight genomic DNA was isolated after 6 days storage at room temperature from stool samples treated with or without CDTA (FIG. 4D).

Total nucleic acids were extracted at T0 and T7 from 200 μL stool aliquots from three donors mixed with the present composition (4% SDS/500 mM Li₂SO₄/10 mM ATA/10 mg/mL PAAc) supplemented with 0-250 mM CDTA or 100 mM EDTA (pH 4.7) using Qiagen's RNeasy® PowerMicrobiome Kit (see Materials & Methods). The final eluate was split into two fractions and treated with either DNAse I or RNAse A. DNA was run on genomic Screentapes, while RNA samples were cleaned-up with Qiagen's RNeasy® MinElute® Cleanup Kit before they were run on RNA screentapes.

For one of the three donors, there was a dramatic reduction in rRNA doublet integrity associated with a drop in RIN in the absence of CDTA at T7 (see FIG. 4E). While not wishing to be bound by theory, the inventors believe that donor 1's stool sample was particularly high in ribonucleases. For the remaining two donors, only a slight drop in RIN was observed in the absence of CDTA (see FIGS. 4F-G). This suggests that donor variability exists in the amounts and types of endogenous nucleases found in stool samples. In contrast, the rRNA doublet appeared intact with high RIN values for all samples in the presence of EDTA, as well as in all other concentrations of CDTA tested (FIGS. 4E-G). In order to account for the variability in RNAse activity seen in donor's samples, chelating agents can be included in the SDS-containing compositions of the present application. In contrast, chelating agents do not appear to be essential for maintaining genomic DNA integrity in stool samples under the experimental conditions (FIGS. 4H-J).

### Example 5. The Role of Buffering Agents in the Stability of Nucleic Acids in Biological Samples

Examples 1 and 2 demonstrate the importance of low pH for the stability of nucleic acids in biological samples. Since PAAc, ATA and CDTA all contribute to the buffering capacity of the present composition, assessing the role of conventional buffering agents (e.g. sodium acetate) is difficult. The present example examines the role of sodium acetate as a buffering agent. The present example also examines the effect of addition of sodium citrate to the composition, which can act as a buffering agent and as noted above also has activity as a chelating agent.

In this example, OMNIgene®⋅GUT Kits (DNA Genotek Inc., Canada) were filled with 4 mL of the compositions of interest (see Table 1, below) and distributed to three healthy donors. The donors dispensed approximately 400-500 mg of stool into each kit and returned the kits to the laboratory where they were stored at room temperature for up to 7 days. 200 μL aliquots were taken and extracted at baseline and after 7 day hold at room temperature with Qiagen's RNeasy® PowerMicrobiome Kit (see Materials & Methods). The final eluate was split into two fractions and treated with either DNAse I or RNAse A. DNA was run on genomic DNA Screentapes, while RNA samples were cleaned-up with Qiagen's RNeasy® MinElute® Cleanup Kit, ran on RNA Screentapes and then visualized on the TapeStation 4200 system (Agilent).

TABLE 1
Test compositions prior to mixing with stool samples.

| | Composition |
|---|---|
| 25 mM Na | 4% SDS, 25 mM NaOAc, 100 mM CDTA, |
| acetate | 500 mM Li₂SO₄, 10 mM ATA, |
| | 10 mg/mL PAAc, pH 4.72 |
| 100 mM Na | 4% SDS, 100 mM NaOAc, 100 mM CDTA, |
| acetate | 500 mM Li₂SO₄, 10 mM ATA, |
| | 10 mg/mL PAAc, pH 4.71 |
| 250 mM Na | 4% SDS, 250 mM NaOAc, 100 mM CDTA, |
| acetate | 500 mM Li₂SO₄, 10 mM ATA, |
| | 10 mg/mL PAAc, pH 4.70 |
| 100 mM Na | 4% SDS, 100 mM Na Citrate, 100 mM CDTA, |
| citrate | 500 mM Li₂SO₄, 10 mM ATA, |
| | 10 mg/mL PAAc, pH 4.71 |

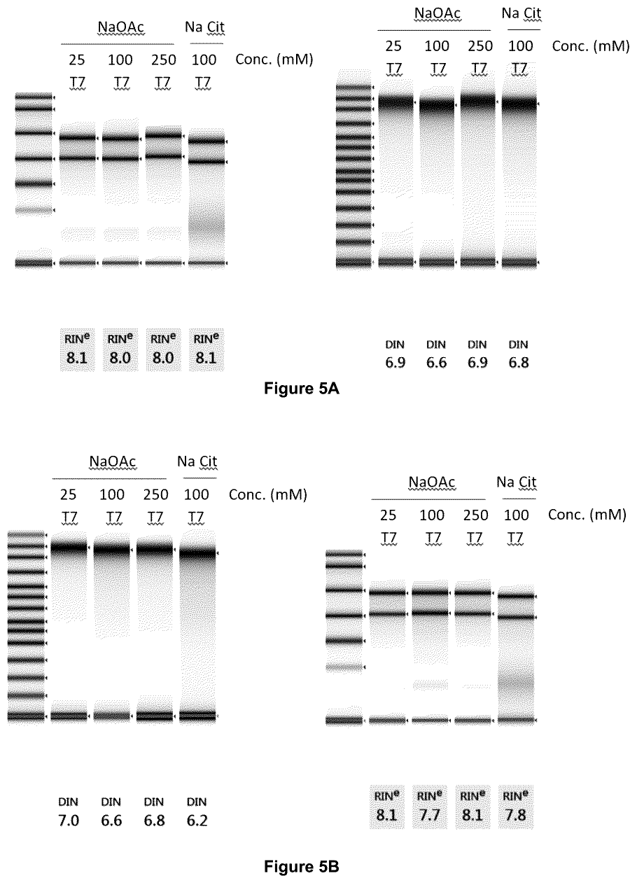

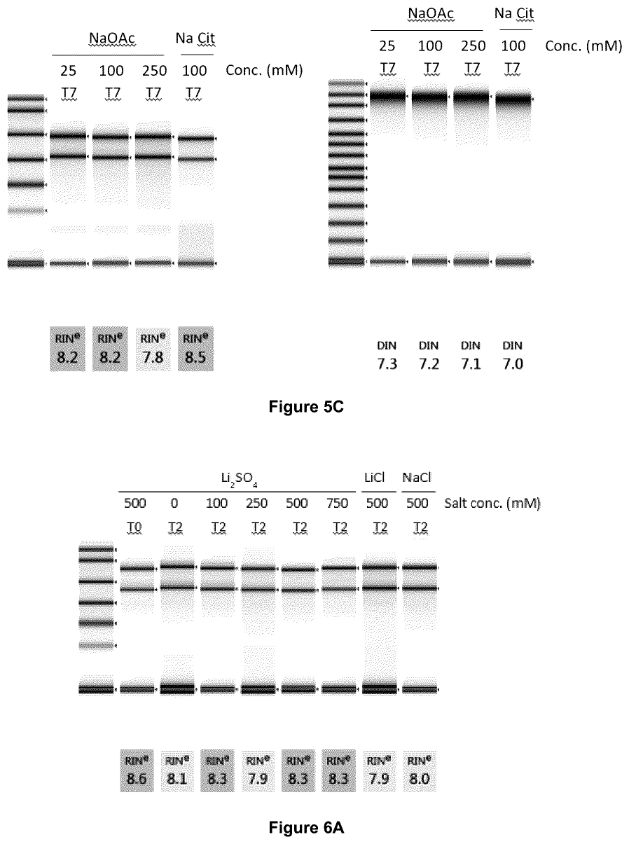

As shown in FIGS. 5A-C, for all three donors, both sodium acetate and sodium citrate, at the concentrations tested, contributed to RNA and DNA stability of stool samples stored at room temperature for 7 days by mainting the pH of the collected samples in our target range (pH 3.8 to 4.9). Therefore, the addition of buffering agent can be beneficial to keep the pH of the composition within the target range.

### Example 6. The Role of Salts on Nucleic Acid Stability in Biological Samples

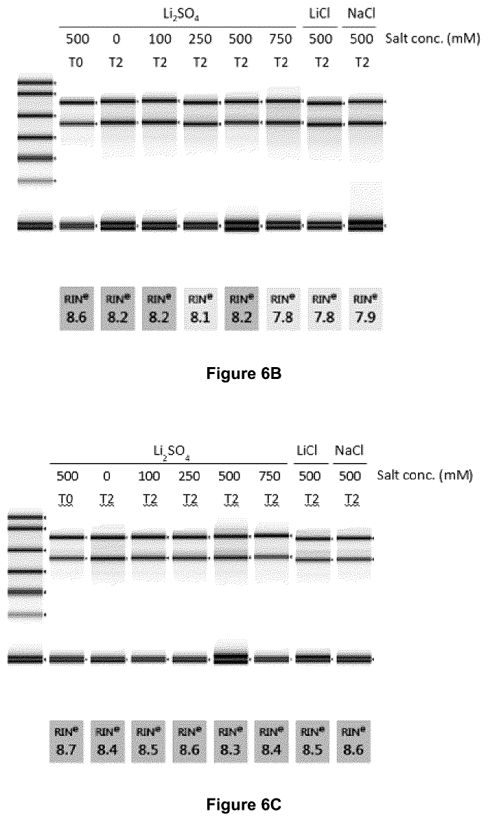

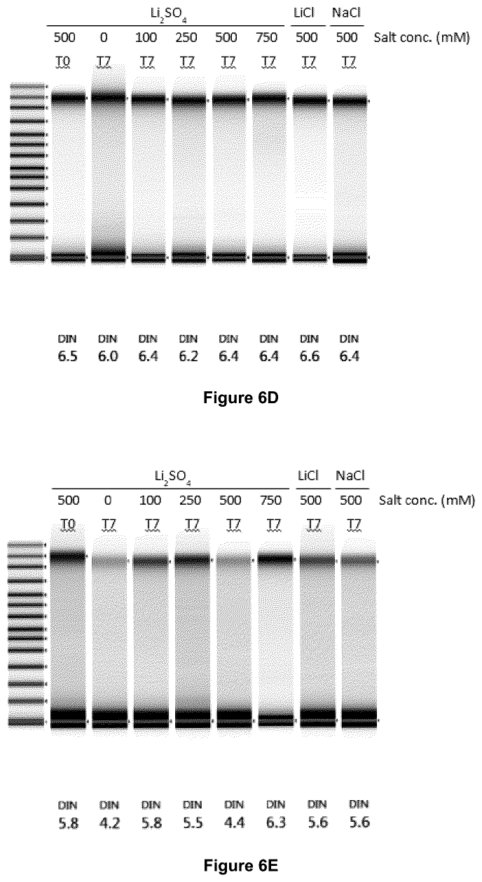

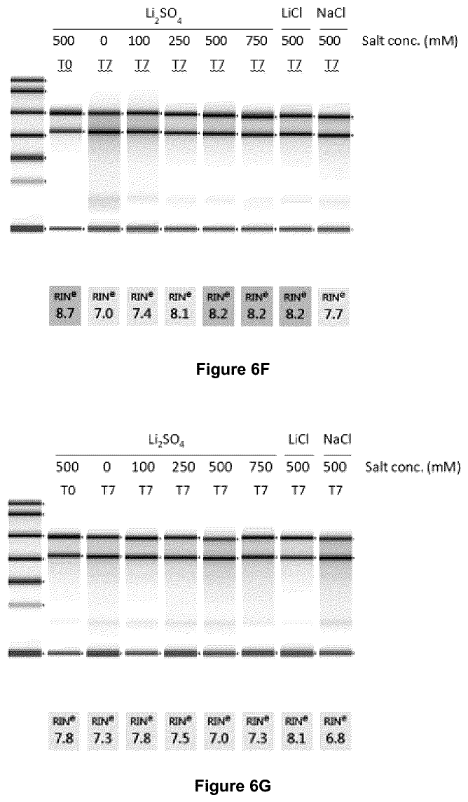

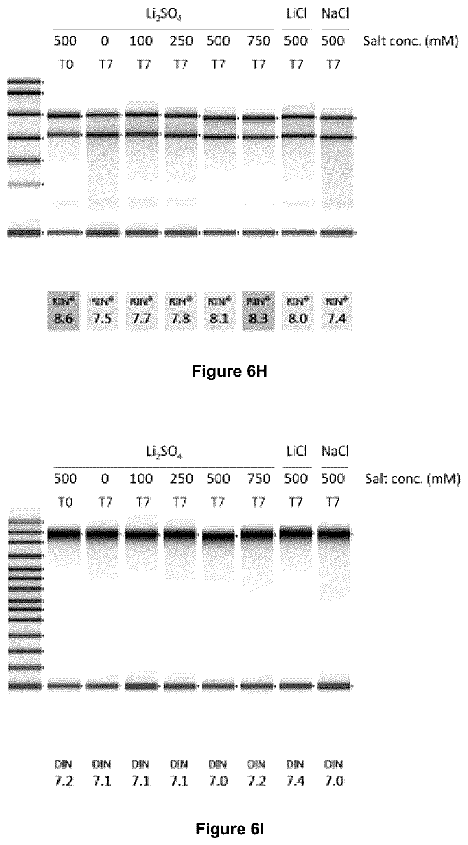

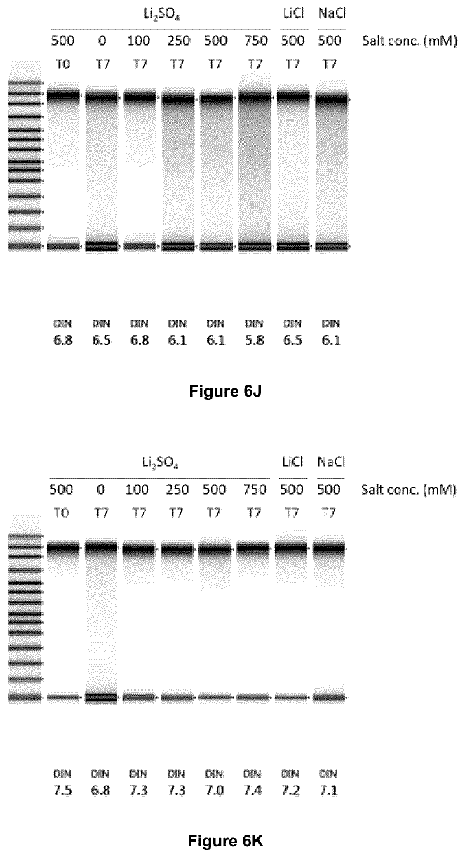

The impact of salts (namely Li₂SO₄, LiCl and NaCl), was tested in the present composition with both saliva and stool samples from healthy donors. Li₂PO₄ and KCl could not be tested due to solubility issues in the present composition. For saliva, samples from three donors were mixed 1:1 with the present composition (4% SDS/100 mM CDTA/10 mM ATA/10 mg/mL PAAc; pH adjusted to 4.6) with increasing concentrations of salts and assessed in the Quickscreen assay (see Materials & Methods). At T0 and T2, spiked RNA was purified from each donor's aliquots using Qiagen's RNeasy® MinElute® Cleanup Kit, run on RNA Screentapes and then visualized on Agilent's TapeStation 4200 system. For all three donors, the rRNA doublet was intact and the RIN was high in the presence and absence of these three salts, Li₂SO₄, LiCl and NaCl (see FIGS. 6A-C), suggesting salts are not necessary for preserving RNA integrity in saliva stored at room temperature.

Endogenous genomic DNA was also purified from two donors' saliva samples using Qiagen's RNeasy® PowerMicrobiome Kit at T0 and T7. The final total nucleic acid eluate was treated with RNAse A, and DNA was run on genomic DNA Screentapes on Agilent's TapeStation 4200 System. For the first donor (see FIG. 6D), genomic DNA was high molecular weight in the absence and presence of salt and the DIN was high, except for a minor decrease in DIN for T7 when salts were completely eliminated from the composition. Similar results were observed for the second donor (see FIG. 6E); however, the intensity of the DNA band was weaker in some conditions, suggesting salts are important for efficient nucleic acid extraction from saliva samples.

In order to test the efficacy of salts in inhibiting nucleases within complex stool samples, the present composition (4% SDS/100 mM CDTA/10 mM ATA/10 mg/mL PAAc) was prepared with increasing concentrations of salt (0-750 mM) and the pH adjusted to 4.6. OMNIgene®⋅GUT Kits (DNA Genotek Inc., Canada) were filled with 4 mL of the compositions of interest and distributed to three healthy donors. The donors dispensed approximately 400-500 mg of stool into each kit and returned the kits to the laboratory where they were stored at room temperature for up to 7 days. After approximately two hours (T0) and seven days (T7), 200 μL aliquots were taken and extracted with Qiagen's RNeasy® PowerMicrobiome Kit (see Materials & Methods). The final eluate was split into two fractions and treated with either DNAse I or RNAse A. DNA was run on genomic DNA Screentapes, while RNA samples were cleaned-up with Qiagen's RNeasy® MinElute® Cleanup Kit, ran on RNA Screentapes and then visualized on the TapeStation 4200 system (Agilent).

Ribosomal RNA (rRNA) bands are largely intact in the presence and absence of salts in stool samples. For samples from all three donors in which salt is omitted, there is a small drop in RIN at T7 (see FIG. 6F-H). This RIN value recovers with increasing additions of salt. Salts do not appear to be critical for the stability of genomic DNA in stool samples (FIGS. 6I-K), but seem to be important for optimal extraction downstream.

### Example 7. The Effect of Guanidinium Salts on Nucleic Acid Stability in Biological Samples

In a previous example, the denaturing agent, sodium dodecyl sulphate (SDS), was shown to be an effective inhibitor of nucleic acid degradation in complex biological samples. In this example, another family of strong denaturing agents, guanidinium salts, were tested for their ability to preserve DNA and RNA in samples stored at room temperature. Guanidinium salts as strong chaotropes and strong denaturants have the ability to denature proteins and decrease enzyme activity while increasing the solubility of hydrophobic molecules.

Within a few hours of collection, saliva from three healthy donors was mixed 1:1 with 2 M guanidinium thiocyanate (GTC) or 4 M guanidinium hydrochloride (GuHCl) plus 50 mM sodium acetate, buffered to pH 4.0, 4.5 and 5.0. At the lab, these samples were spiked with Francisella philomiragia total RNA (Quickscreen, see Materials & Methods) and stored at room temperature for 3 days. At T=0 and T=3 days an aliquot of each sample was purified using RNeasy® MiniElute® Cleanup Kit (Qiagen) and then run on RNA screentapes on the TapeStation 4200 system (Agilent).

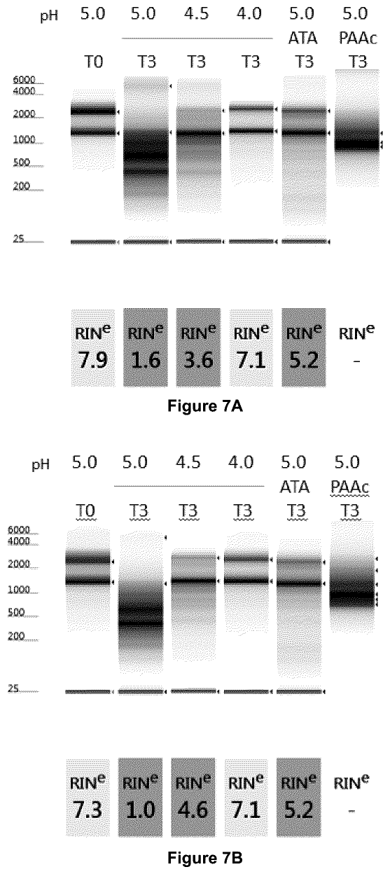

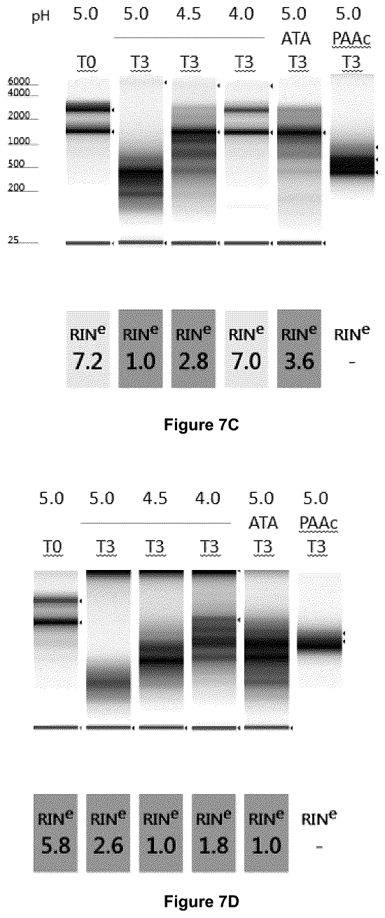

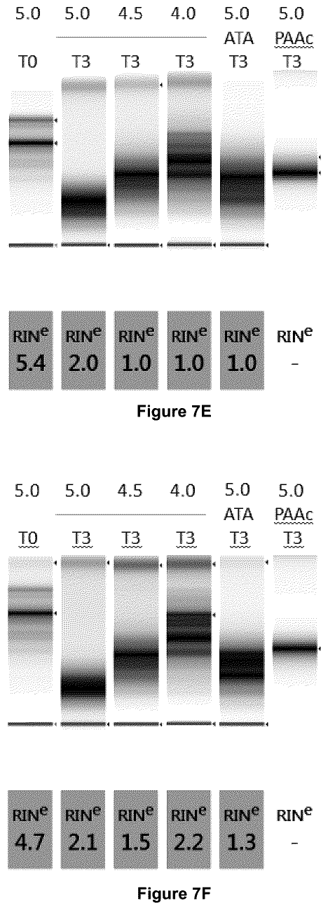

For both guanidinium salts and all saliva donors tested, there was a surprising improvement in RNA stability and corresponding increase in RIN as the pH decreased from 5.0 to 4.5 and then 4.0 (see FIGS. 7A-F). The addition of ATA (10 mM) to the guanidinium salts-based samples also led to a significant improvement in RNA stability. PAAc (10 mg/mL), while not as effective as ATA, also led to an improvement in RNA stability. Overall, GTC appears to be more effective than GuHCl for stabilizing RNA at low pH (compare FIGS. 7A-C to FIGS. 7D-F).

### Example 8. The Effect of Guanidinium Salts on Endogenous Nuclease Activity in Biological Samples

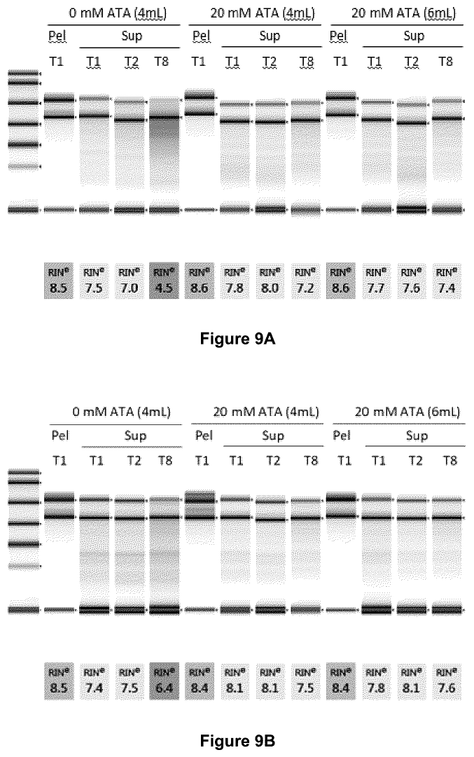

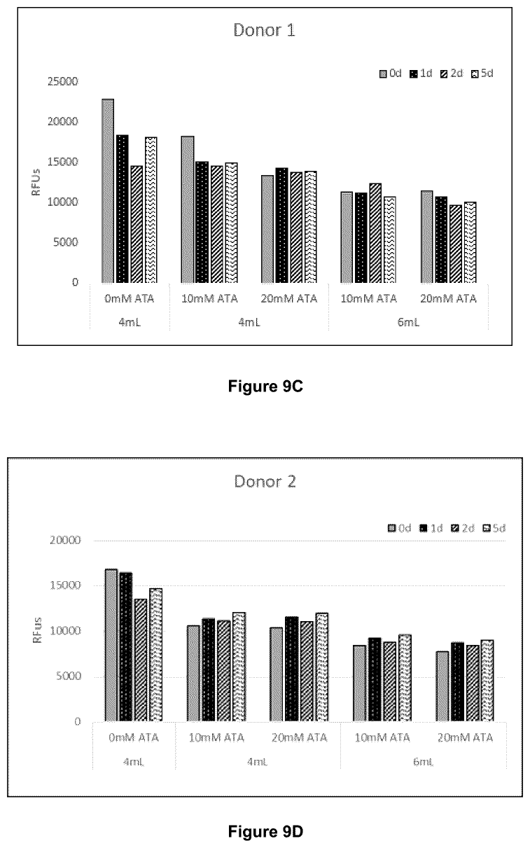

In a previous example, guanidinium salts were shown to improve RNA stability at low pH (see Example 7). In an attempt to quantify RNAse activity in biological samples stored under different conditions, the RNAseAlert® assay (IDT; see Materials & Methods) was adapted for use with biological samples. Specifically, GTC- and GuHCl-based compositions (outlined in Tables 2 and 3 below) were mixed 1:1 with saliva from healthy donors and then incubated at room temperature for approximately 1 hour prior to quantitation of RNAse activity using the adapted RNAseAlert® assay (see FIGS. 9A and B).

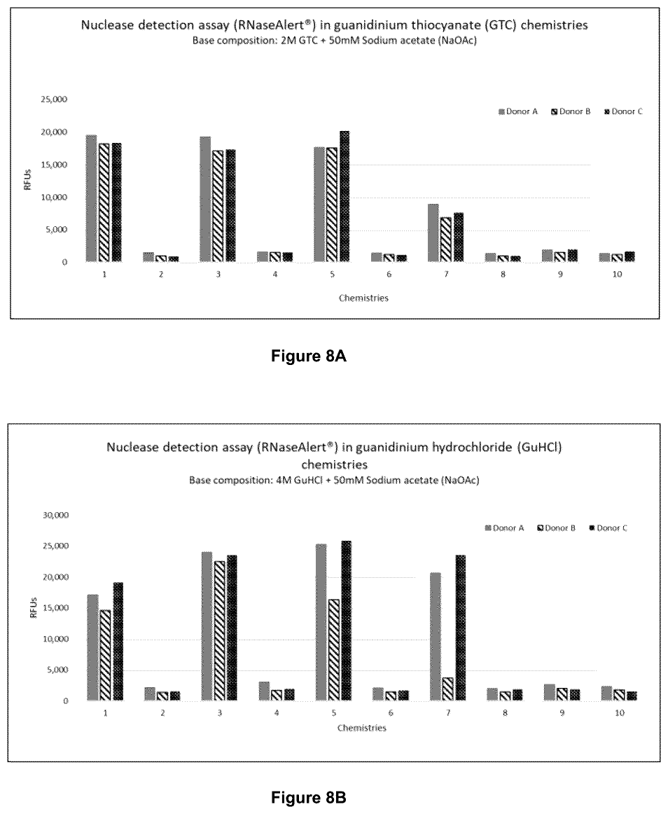

At pH 4.0, 4.5 and 4.7, high RNAse activity (>14,000 RFU) was detected in saliva samples from all donors collected in guanidinium thiocyanate (FIG. 8A) or guanidinium hydrochloride (FIG. 8B) in the absence of ATA and PAAc. The addition of ATA (5 mM final) to these guanidinium salt-based compositions dramatically reduced RNAse activity in each donor's sample (FIG. 8A and B). At a pH of 4.7, PAAc also reduced RNAse activity, but to a lesser degree than ATA (FIG. 8A and B). The addition of both ATA and PAAc dramatically reduced RNAse activity to low levels seen with ATA alone. The RNAseAlert® assay may not be sensitive enough to distinguish a difference in RNAse activity between ATA- and PAAc-treated samples at such low RFU values. The addition of CDTA to these guanidinium-based salts had minimal effect on RNAse activity (FIG. 8A and B) when ATA was present. Hence, ATA is essential for reducing endogenous RNAse activity in saliva samples treated with guanidinium salts.

TABLE 2
Description of guanidinium thiocyanate-based compositions mixed
1 to 1 with saliva samples for nuclease activity assessment.

| | Base | RNAse | Additional | |
|---|---|---|---|---|
| | Chemistry | inhibitors | components | pH |
| 1 | 2M GTC, | | | 4.0 |
| 2 | 50 mM NaOAc | 10 mM ATA | | 4.0 |
| 3 | | | | 4.5 |
| 4 | | 10 mM ATA | | 4.5 |
| 5 | | | | 4.7 |
| 6 | | 10 mM ATA | | 4.7 |
| 7 | | 10 mg/mL PAAc | | 4.7 |
| 8 | | 10 mM ATA + | | 4.7 |
| | | 10 mg/mL PAAc | | |
| 9 | | 10 mM ATA | 100 mM CDTA | 4.7 |
| 10 | 2M GTC | 10 mM ATA | 100 mM CDTA | 4.7 |

TABLE 3
Description of guanidinium hydrochloride-based compositions mixed
1 to 1 with saliva samples for nuclease activity assessment.

| | Base | RNase | Additional | |
|---|---|---|---|---|
| | Chemistry | inhibitors | components | pH |
| 1 | 4M GuHCl, | | | 4.0 |
| 2 | 50 mM NaOAc | 10 mM ATA | | 4.0 |
| 3 | | | | 4.5 |
| 4 | | 10 mM ATA | | 4.5 |
| 5 | | | | 4.7 |
| 6 | | 10 mM ATA | | 4.7 |
| 7 | | 10 mg/mL PAAc | | 4.7 |
| 8 | | 10 mM ATA + | | 4.7 |
| | | 10 mg/mL PAAc | | |
| 9 | | 10 mM ATA | 100 mM CDTA | 4.7 |
| 10 | 4M GuHCl | 10 mM ATA | 100 mM CDTA | 4.7 |

### Example 9. Endogenous RNA Stability and RNase Levels in Stool Samples Collected in Different Volumes of the Present Composition

In the present example, 400-500 mg stool samples from two healthy donors were collected into OMNIgene®⋅GUT Kits (DNA Genotek Inc., Canada) filled with 4 mL or 6 mL of the compositions of interest (see Table 4) and stored at room temperature for up to 8 days. To prevent excessive bubbling during mixing of the samples at collection, 0.1% Antifoam A was also added to the composition in each kit.

TABLE 4
Compositions tested with stool samples.

| Condition | Composition Tested |
|---|---|
| 0 mM ATA | 4% SDS/100 mM CDTA/500 mM |
| | Li₂SO₄, pH 4.9 |
| 10 mM ATA | 4% SDS/100 mM CDTA/250 mM |
| | Li₂SO₄/10 mM ATA/10 mg/mL |
| | PAAc, pH 4.5 |
| 20 mM ATA | 4% SDS/100 mM CDTA/250 mM |
| | Li₂SO₄, 20 mM ATA/10 mg/mL |
| | PAAc pH 4.5 |

Following 1 day at room temperature, an aliquot of each stool sample was removed, and the pellet and supernatant fractions were isolated by centrifugation. Endogenous RNA was extracted from each fraction using Qiagen's RNeasy® PowerMicrobiome kit. RNA was also extracted from isolated supernatants incubated at room temperature for 2 and 8 days. Purified RNA was run on RNA screentapes and then visualized on the TapeStation 4200 system (Agilent) (see FIGS. 9A-B). In the absence of ATA, there is a small reduction in RIN with increasing time at room temperature. In contrast, RIN values are relatively stable over time in aliquots treated with ATA. In both cases, the RNA doublet remains visible on the RNA screentapes. Stool collected into 4 mL or 6 mL of the present composition with ATA performed similarly in terms of RNA doublet stability (see FIGS. 9A-B).

To quantify RNAse activity in stool samples mixed with the present composition (see Table 4) the RNAseAlert® assay (IDT; see Materials & Methods) was employed. In the absence of ATA (FIGS. 9C-D), consistently high RNAse activity (>13,000 RFU) was detected in stool samples from both donors at T0 and after 1, 3 or 5 days. With increasing concentration of ATA from 10 mM to 20 mM there was a reduction in RNAse activity in samples from both donors. RNAse activity was reduced even further by increasing the ratio of chemistry to sample (FIG. 9C-D).

### Example 10. RNA Stability, DNA Stability and RNAse Levels in Stool Samples Stored in the Present Composition for 1 day at Room Temperature

In the present example, 400-500 mg of stool from three healthy donors was collected into OMNIgene® GUT Kits (DNA Genotek Inc., Canada) containing 4 mL of stabilizing solution comprised of 1) 8% SDS, 250 mM Li₂SO₄, 100 mM CDTA, 20 mM ATA, 10 mg/mL PAAc, pH 4.3 or 2) 8% SDS, 250 mM Li₂SO_{4, 100} mM CDTA, pH 6.5. To prevent excessive bubbling during mixing of the samples during collection, 0.1% Antifoam A was also added to the composition in each kit.

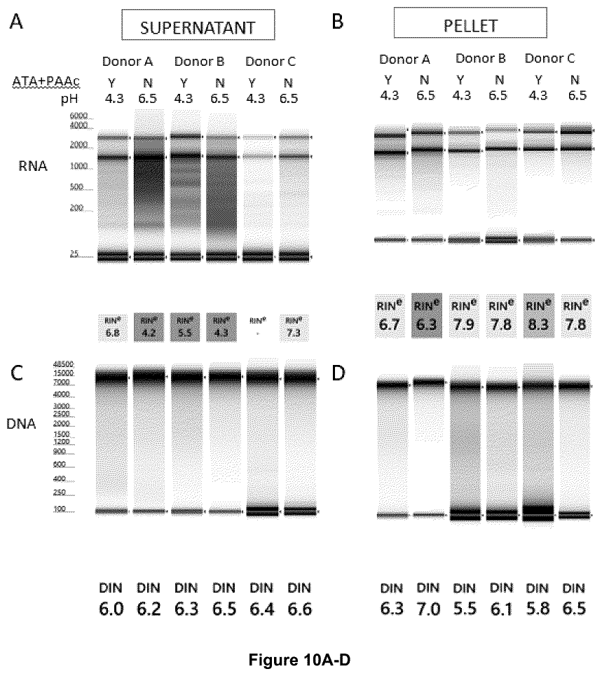

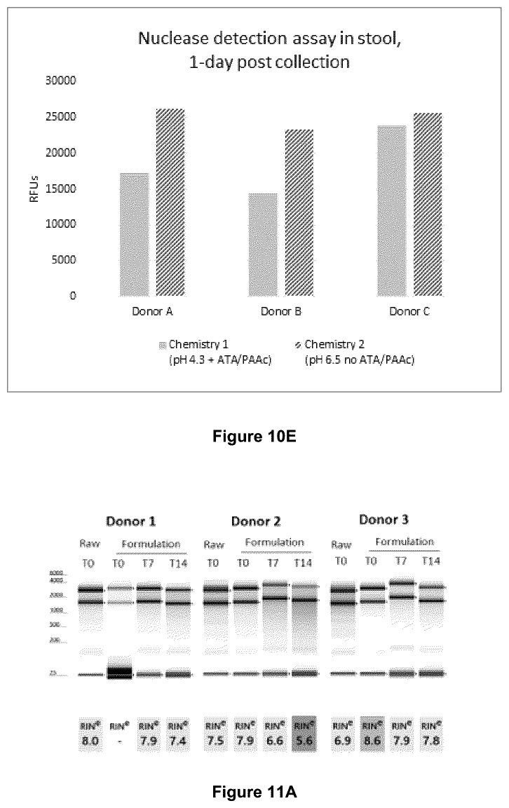

Following one day at room temperature, supernatant and pellet fractions were isolated from an aliquot of each stool sample by centrifugation. Subsequently, endogenous total nucleic acids were extracted from each fraction using Qiagen's RNeasy® PowerMicrobiome kit (see Materials & Methods). An aliquot of each elution was treated with DNAse and RNA was then purified with the RNeasy® MinElute Clean up kit, and run on RNA screentapes on the TapeStation 4200 system (Agilent). Conversely, the remainder of the elution was treated with RNAse A, and then the DNA was run on genomic DNA screentapes (see FIG. 10A-D).

Lowering pH to 4.3 and the addition of ATA and PAAc had a positive impact on RNA stability in the supernatant fractions as visualized by intact RNA doublets and higher RIN values, compared to the samples collected in a composition at pH 6.5 that lacked ATA and PAAc (FIG. 10A). In the pellet fractions (FIG. 10B), the RNA doublet was intact in both compositions and RIN values were fairly stable.

As shown in FIG. 10C-D, high molecular weight genomic DNA was recovered from the supernatant and pellet fractions for all three donors regardless of the composition. There was a slight decrease in DIN observed in samples stored in 8% SDS, 250 mM Li₂SO₄, 100 mM CDTA, 20 mM ATA, 10 mg/mL PAAc, pH 4.3, compared to samples stored in 8% SDS, 250 mM Li₂SO₄, 100 mM CDTA, PH 6.5.

To quantify RNAse activity in stool samples mixed with the present composition the RNAseAlert® assay (IDT; see Materials & Methods) was employed. Lowering pH to 4.3 and the addition of ATA and PAAc had a negative impact on RNAse activity, compared to the samples collected in a composition at pH 6.5 that lacked ATA and PAAc (see FIG. 10E). This demonstrates that the present formulation is effective at inhibiting nuclease activity.

### Example 11. The Present Composition Maintains Nucleic Acid Stability and RNA Profiles of Stool Samples Stored for up to 14 Days at Room Temperature

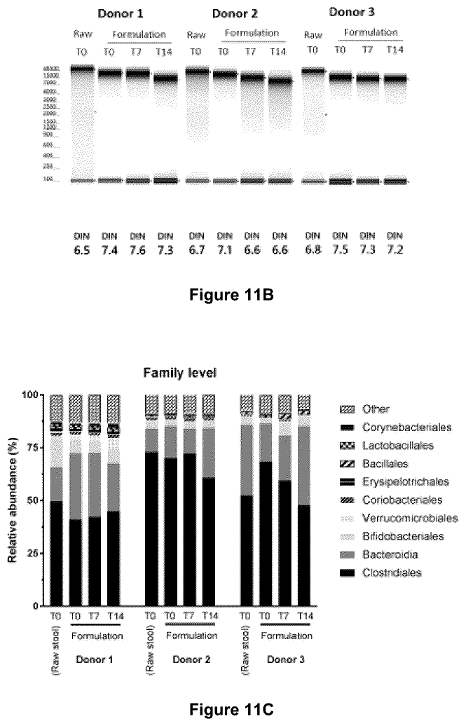

In the present example, 400-500 mg stool samples from three healthy donors were collected into OMNIgene®⋅GUT Kits (DNA Genotek Inc., Canada) filled with 4 mL of 4% SDS, 100 mM CDTA, 500 mM Li₂SO₄, 10 mM ATA and 10 mg/mL PAAc at pH 4.7. To prevent excessive foaming during homogenization, 0.1% Antifoam A was also added to the composition in each kit. Total nucleic acids were extracted from each stool sample at baseline (T0, 2-3 h post collection) and after 7 and 14 days incubation at room temperature using Qiagen's RNeasy® PowerMicrobiome kit. Total nucleic acids were also extracted from matching aliquots of raw stool that were immediately frozen on dry ice and transported back to the laboratory for extraction. An aliquot of the eluate was treated with DNAse and then purified with Qiagen's RNeasy® MinElute® Cleanup Kit. Purified RNA was then run on RNA screentapes and visualized using the TapeStation 4200 system (Agilent) (see FIG. 11A). Similarly, another aliquot from the total nucleic acid extraction was treated with RNAse A, and the resulting genomic DNA fraction was then run on a DNA screentape and visualized using the TapeStation 4200 system (Agilent) (see FIG. 11B).

For all donors RNA integrity/quality was maintained over time in the present composition and similar to quality seen for the raw sample at baseline, despite a small drop in RIN value in one of the donors (FIG. 11A). DNA was also stable over time and high molecular weight DNA was recovered from both raw stool and samples collected in the present composition for all time point and all three donors (FIG. 11B).

To check the RNA profile stability of stool samples collected in the present formulation, metatranscriptomics sequencing was performed (see Materials & Methods) on the RNA samples extracted at baseline (T0), and following storage at room temperature for 7 to 14 days. Sequencing results show that the taxonomic profiles of the three stool samples collected in 4 mL of 4% SDS, 100 mM CDTA, 500 mM Li₂SO₄, 10 mM ATA and 10 mg/mL PAAc at pH 4.7, are comparable to the profile of the matching raw samples and stable during storage within the compositions at room temperature for 7 or 14 days (FIG. 11C). Profile stability was maintained at phylum, family (FIG. 11C), genus and species levels (data not shown).

### Example 12. Nucleic Acid Stability in Stool Samples Stored Under Ambient Conditions

In order to simulate the conditions observed during the shipping of stool samples from the point of collection to the laboratory three scenarios were tested: 1) samples were kept at room temperature for 12 days; 2) samples were subjected to 37° C. for up to 3 days; and 3) samples were exposed to three cycles of freezing at −20° C., followed by exposure to either 37° C. or 50° C. (with a minimum incubation of 3 hours at each temperature) over the course of 5 days.

In this study, 400-500 mg stool samples from 12 healthy donors were collected into OMNIgene®⋅GUT Kits (DNA Genotek Inc., Canada) filled with 4 mL of 4% SDS, 100 mM CDTA, 500 mM Li₂SO₄, 10 mM ATA and 10 mg/mL PAAc at pH 4.7. To prevent excessive foaming during homogenization, 0.1% Antifoam A was also added to the composition in each kit. Aliquots were taken at baseline from the main collected tubes and either kept at room temperature for 12 days, 37° C. for up to 3 days, or subjected to three cycles of freeze/thaw (−20° C. to +37° C. or −20° C. to +50° C.) over the course of 5 days. At the appropriate time, total nucleic acids were extracted from each aliquot using Qiagen's RNeasy® PowerMicrobiome kit (see Materials & Methods). An aliquot of the eluate was treated with DNAse and then purified with Qiagen's RNeasy® MinElute® Cleanup Kit. Purified RNA was then run on RNA screentapes and visualized using the TapeStation 4200 system (Agilent). Similarly, another aliquot from the total nucleic acid extraction was treated with RNAse A, and the resulting genomic DNA fraction was then run on a DNA screentape and visualized using the TapeStation 4200 system (Agilent).

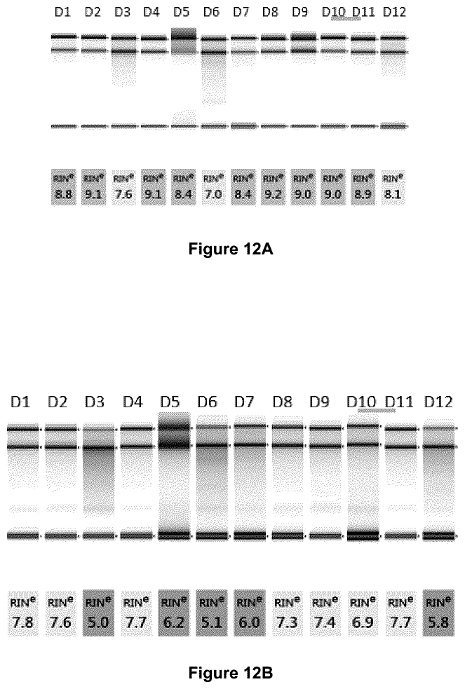

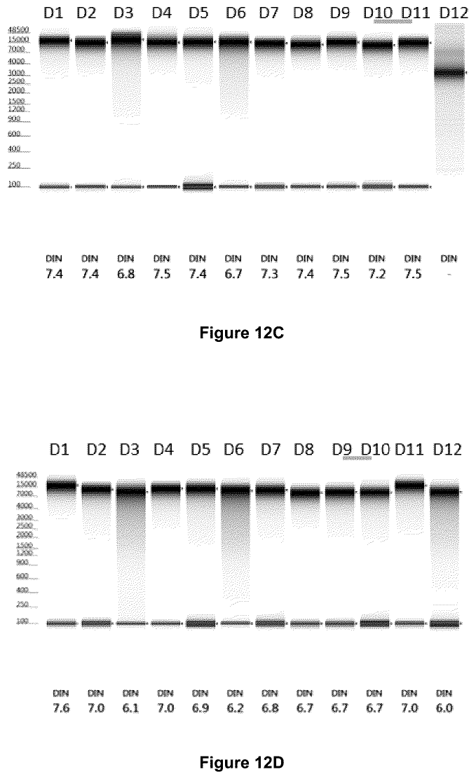

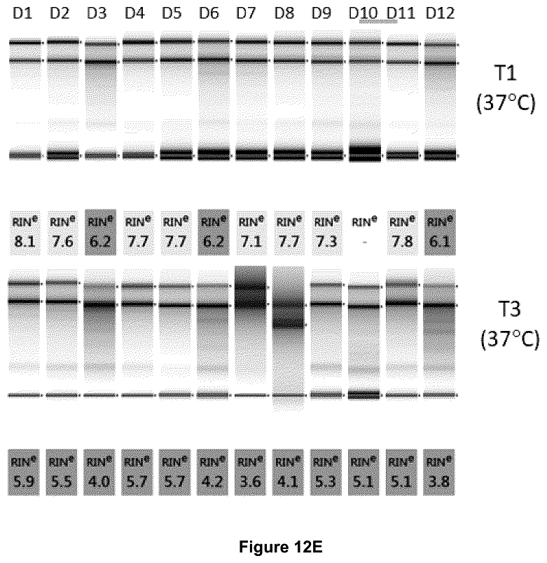

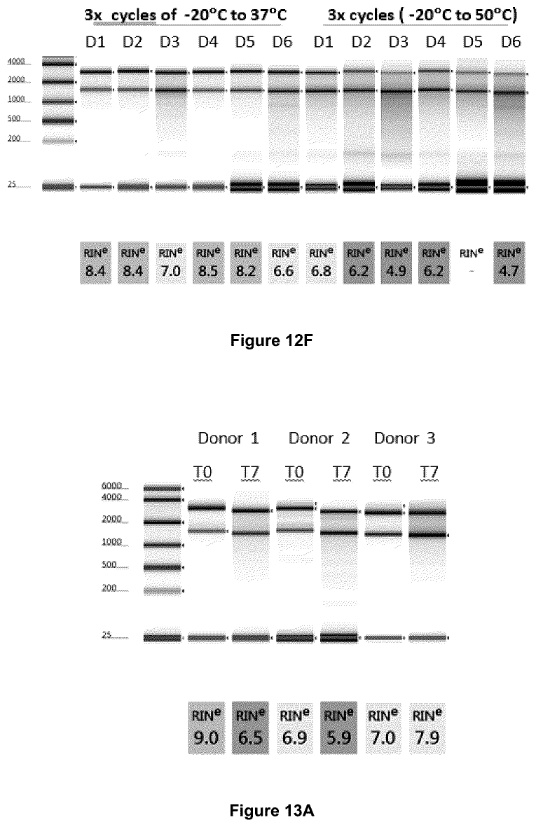

Endogenous ribosomal RNA doublet was intact in stool samples from all 12 donors at T0 (see FIG. 12A) and following incubation for 12 days (FIG. 12B) at room temperature in the present composition. Similarly, high molecular weight endogenous DNA was recovered from all 12 donors at T0 (FIG. 12C) and following incubation for 12 days (FIG. 12D) at room temperature. Following exposure to 37° C. for 1 and 3 days, endogenous RNA doublet for all 12 donors was largely intact in stool samples collected into the present composition (FIG. 12E). Finally, endogenous stool RNA from all donors was preserved in our composition at T5 following three cycles of freeze/thaw. Hence, the present composition stabilizes nucleic acids in stool samples during the extreme temperature conditions that can be encountered during transport.

### Example 13. Nucleic Acid Stability in Stool Samples from Infants

Compared to adult stool, infant stool is comprised of different bacterial profiles and has a lower biomass (Milani et al., 2017). In this study, 400-500 mg stool samples from 3 healthy infants (aged between 1 and 8 months) were collected from diapers into OMNIgene®⋅GUT Kits (DNA Genotek Inc., Canada) filled with 4 mL of 4% SDS, 100 mM CDTA, 500 mM Li₂SO₄, 10 mM ATA and 10 mg/mL PAAc at pH 4.7. To prevent excessive foaming during homogenization, 0.1% Antifoam A was also added to the composition in each kit. Aliquots were taken at baseline (T0) and following 7 days at room temperature (T7) from the main collected tubes. Total nucleic acids were extracted from each aliquot using Qiagen's RNeasy® PowerMicrobiome kit (see Materials & Methods). An aliquot of the eluate was treated with DNAse and then purified with Qiagen's RNeasy® MinElute® Cleanup Kit. Purified RNA was then run on RNA screentapes and visualized using the TapeStation 4200 system (Agilent). Similarly, another aliquot from the total nucleic acid extraction was treated with RNAse A, and the resulting genomic DNA fraction was then run on a DNA screentape and visualized using the TapeStation 4200 system (Agilent).

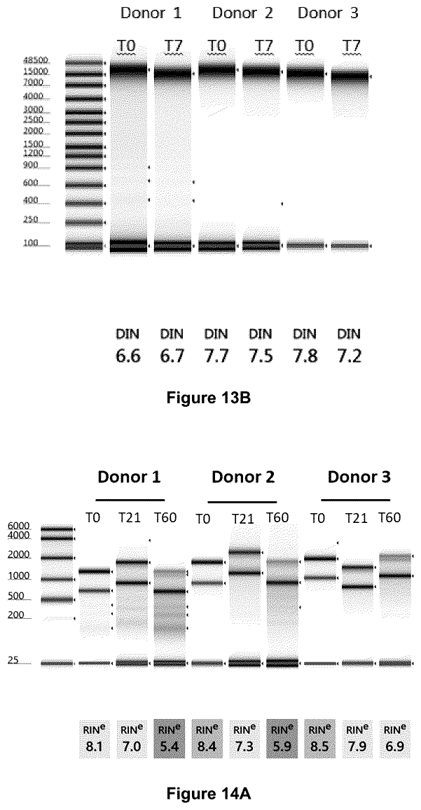

For all three infant donors, endogenous ribosomal RNA doublet was intact in stool samples stored in the present composition for up to 7 days at room temperature (FIG. 13A). Similarly, endogenous DNA was high molecular weight in these stool samples at T0 and T7 (FIG. 13B). Hence, the present composition effectively stabilizes nucleic acids in infant stool samples stored at room temperature.

Milani C, Duranti S, Bottacini F, Casey E, Turroni F, Mohony J, Belzer C, Palacio SD, Montes SA, Mancabelli L, Lugli GA, Rodriguez JM, Bode L, de Vos W, Gueimonde M, Margolles A, van Sinderen D and Venture M. The first microbial colonizers of the human gut: Composition, Activities, and health implications of the infant gut microbiota (2017) Microbiol Mol Biol Rev 81(4): e00036-17.

### Example 14. The Present Composition Maintains Viral and Human RNA Stability and Bacterial DNA and RNA Stability Following Incubation at Room Temperature for Up to 60 Days

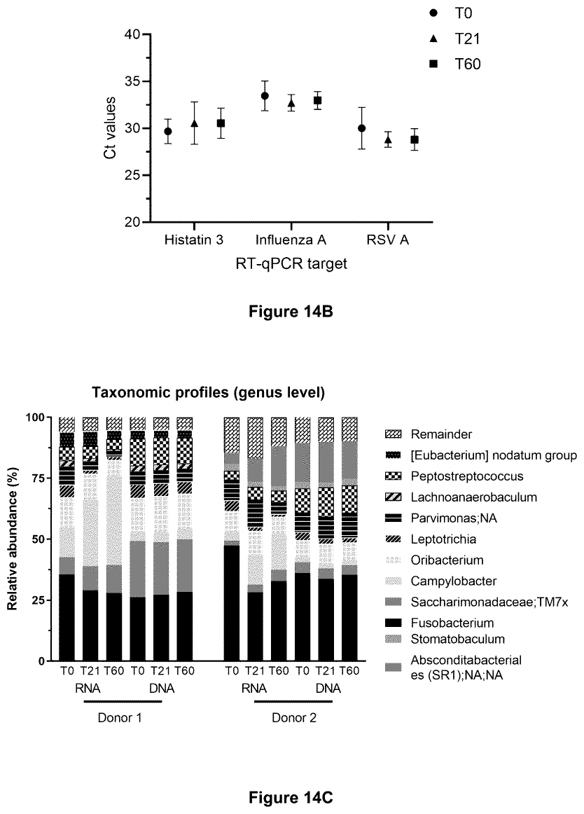

RSV A and Influenza A viruses were ordered from ATCC (Cat. No. VR-26 & VR-1894 respectively). In the present example, 1 mL saliva was collected from 12 healthy donors, Influenza A was spiked @6587 TCID50/mL and RSV A was spiked @1000 TCID50/mL in the saliva samples, and the spiked samples were mixed 1:1 with 1 mL of 4% SDS, 100 mM CDTA, 500 mM Li₂SO₄, 10 mM ATA and 10 mg/mL PAAc at pH 4.7. Total nucleic acids were extracted from each sample at baseline (T0, ˜1 h post collection) and after 21 and 60 days incubation at room temperature using Qiagen's RNeasy® PowerMicrobiome kit or ThermoFisher MagMax™ viral pathogen nucleic acid extraction kit. An aliquot of the PowerMicrobiome eluates were treated with DNAse and then purified with Qiagen's RNeasy® MinElute® Cleanup Kit. Purified total RNA was then run on RNA screentapes and visualized using the TapeStation 4200 system (Agilent) (see FIG. 14A). Despite a small drop in RIN value after 60 days storage, RNA integrity/quality was maintained over time in the present composition for all donors and similar to that of the sample at baseline.

Stability of human and viral RNA was also assessed over time using RT-qPCR assays targeting the human histatin 3 mRNA, as well as the viral Influenza A and respiratory syncytial virus A (RSV A). Human and viral RNA levels were stable over time (no changes in the Ct values) and comparable to baseline (FIG. 14B).

To check bacterial DNA and RNA profile stability of samples collected in the present composition, 16S amplicon sequencing was performed (see Materials and Methods) on the DNA and RNA samples extracted at baseline (T0) and following storage at room temperature for 21 and 60 days. Sequencing results show that the DNA and RNA taxonomic profiles of samples mixed 1:1 with 4% SDS, 100 mM CDTA, 500 mM Li₂SO₄, 10 mM ATA and 10 mg/mL PAAc at pH 4.7, are stable over time and comparable to the profile of the matching sample at baseline (FIG. 14C).

### REFERENCES

Anderson EL, Li W, Klitgord N, Highlander SK, Dayrit M, Seguritan V, Yooseph S, Biggs W, Venter JC, Nelson KE and Jones MB. A robust ambient temperature collection and stabilization strategy: Enabling worldwide functional studies of the human microbiome. Sci. Rep. 6, 31731; doi: 10.1038/srep31731 (2016).
Backhed F, Ding H, Wang T, Hooper LV, Koh GY, Nagy A, Semenkovich CF and Gordon JI. The gut microbiota as an environmental factor that regulates fat storage. PNAS 101(44): 15718-15723; doi: 10.1073/pnas.0407076101 (2004).
Barra GB, Santa Rita TH, Vasques JA, Chianca CF, Nery LFA, Costa SSS. EDTA-mediated inhibition of DNases protects circulating cell-free DNA from ex vivo degradation in blood samples. Clinical Biochemistry 48 (2015): 976-981.
Bechhofer DH and Deutscher MP. Bacterial ribonucleases and their roles in RNA metabolism. Crit Rev Biochem Mol Biol (2019) 54(3): 242-300. Doi: 10.1080/10409238.2019.1651816.
Belstrom D. The salivary microbiota in health and disease. J Oral Microbiol (2020): 12: 1723975. Doi: 10.1080/20002297.2020.1723975.
Blumberg DD. Creating a ribonuclease-free environment (1987) Methods in Enzymology (152): 20-24.
Carabotti M, Scirocco A, Maselli MA and Severi C. The gut-brain axis: interactions between enteric microbiota, central and enteric nervous systems. Ann Gastroenterol (2015); 28(2): 203-209.
Cardona S, Eck A, Cassellas M, Gallart M, Alastrue C, Dore J, Azpiroz F, Roca J, Guarner F and Manichanh C. Storage conditions of intestinal microbiota matter in metagenomic analysis. BMC Microbiology (2012) 12: 158. Doi: 10.1186/1471-2180-12-158.
Carroll IM, Ringel-Kulka T, Siddle JP, Klaenhammer TR and Ringel Y (2012). Characterization of the fecal microbiota using high-throughput sequencing reveals a stable microbial community during storage. PLOS ONE 7(10): e46953. Doi:10.1371/journal.pone.0046953.
Chen Z, Ling J and Gallie DR. RNase activity requires formation of disulfide bonds and is regulated by the redox state (2004). Pant Molecular Biology 55: 83-96.
Choo JM, Leong LE and Rogers GB. Sample storage conditions significantly influence faecal microbiome profiles (2015) Sci. Rep. 5, 16350. Doi: 1038/srep16350.
Clarke TB, Davis KM, Lysenko ES, Zhou AY, Yu Y and Weiser JN. Recognition of peptidoglycan from the microbiota by Nod1 enhances systemic innate immunity (2010) Nat Med 16(2):228-231. Doi: 10.1038/nm.2087.
Commichau FM, Rothe FM, Herzberg C, Wagner E, Hellwig D, Lehnik-Habrink M, Hammer E, Volker U and Stulke J. Novel activities of glycolytic enzymes in Bacillus subtilis (2009) Molecular & Cellular Proteomics 8(6): 1350-1360. Doi: 10.1074/MCP.M800546-MCP200.
Condon C. RNA processing and degradation in Bacillus subtilis (2003) Microbiol Mol Biol Rev 67(2): 157-174. Doi: 10.1128/MMBR.67.2.157-174.2003.
Dethlefsen L, McFall-Ngai M and Relman DA. An ecological and evolutionary perspective on human-microbiome mutualism and disease (2007) Nature 449: 811-818. Doi: 10.1038/nature06245.
Dupureur CM. Roles of metal ions in nucleases (2008) Current Opinion in Chemical Biology 12: 250-255.
Earl CC, Smith MT, Lease RA and Bundy BC. Polyvinylsulfonic acid: A low-cost RNAse inhibitor for enhanced RNA preservation and cell-free protein translation (2018). Bioengineered 9(1): 90-97. Doi: 10.1080/21655979.2017.1313648.
Feng Q, Liang S, Jia H, Stadlmayr A, Tang L, Lan Z, Zhang D, Xia H, Xu X, Jie Z, Su L, Li X, Li X, Li J, Xiao L, Huber-Schonauer U, Niederseer D, Xu X, Al-Aama JY, Yang H, Wang J, Kristiansen K, Arumugam M, Tilg J, Datz C and Wang J. Gut microbiome development along the colorectal adenoma-carcinoma sequence (2014) Nature Communications 6: 6528. Doi: 10.1038/ncomms7528.
Flores R, Shi J, Yu G, Ma B, Ravel J, Goedert JJ and Sinha R. Collection media and delayed freezing effects on microbial composition of human stool (2015) Microbiome 3:33. Doi: 10.1186/s40168-015-0092-7.
Fouhy F, Deane J, Rea MC, O'Sullivan O, Ross RP, O'Callaghan G, Plant BJ and Stanton C. The effects of freezing on faecal microbiota as determined using MiSeq sequencing and culture-based investigations (2015) PLOS ONE 10(3): e0119355. Doi: 10.1371/journal.pone.0119355.
Guo Y, Li S-H, Kuang Y-S, He J-R, Lu J-H, Luo B-J, Jiang F-J, Liu Y-Z, Papasian CJ, Xia H-M, Deng H-W and Qiu X. Effect of short-term room temperature storage on the microbial community in infant fecal samples (2016) Scientific Reports 6: 26648. Doi: 10.1038/srep26648.
Hansen CHF, Andersen LSF, Krych L, Metzdorff SB, Hasselby JP, Skov S, Nielsen DS, Buschard K, Hansen LH and Hansen AK. Mode of delivery shapes gut colonization pattern and modulates regulatory immunity in mice (2014) J Immunol 193: 1213-1222. Doi: 10.4049/jimmunol. 1400085.
Hedin C, van der Gast C, Rogers GB, Cuthbertson L, McCartney S, Stagg AJ, Lindsay JO and Whelan K. Siblings of patients with Crohn's disease exhibit a biologically relevant dysbiosis in mucosal microbial metacommunities (2015) Gut 65: 944-953. Doi: 10.1136/gutjnl-2014-308896.
Huang K-W, Hsu K-C, Chu L-Y, Yang J-M, Yuan HS and Hsiao Y-Y. Identification of inhibitors for the DEDDh family of exonucleases and a unique inhibition mechanism by crystal structure analysis of CRN-4 bound with 2-morpholin-4-ylethanesulfonate (MES) (2016) J Med Chem 59: 8019-8029. Doi: 10.1021/acs.jmedchem.6b00794.
Keyel PA. DNAses in health and disease (2017) Developmental Biology 429: 1-11. Doi: 10.1016/j.ydbio.2017.06.028.
Kim B-M, Schultz LW and Raines RT. Variants of ribonuclease inhibitor that resist oxidation (1999) Protein Science 8: 430-434.
Kime L, Vincent HA, Gendoo DMA, Jourdan SS, Fishwick CWG, Callaghan AJ and McDowall KJ. The first small-molecule inhibitors of members of the ribonuclease E family (2015) Scientific Reports 5: 8028. Doi: 10.1038/srep08028.
Koboziev I, Webb CR, Furr KL and Grisham MB. Role of the enteric microbiota in intestinal homeostasis and inflammation (2014) Free Radic Biol Med 0: 122-133. Doi: 10.1016/j.freeradbiomed.2013.11.008.
Koenig JE, Spor A, Scalfone N, Fricker AD, Stombaugh J, Knight R, Angenent LT and Ley RE. Succession of microbial consortia in the developing infant gut microbiome (2010) PNAS 108(1): 4578-4585. Doi: 10.1073/pnas.1000081107.
Kostic AD, Gevers D, Siljander H, Vatanen T, Hyotylainen T, Hamalainen A-M, Peet A, Tillmann V, Poho P, Mattila I, Lahdesmaki H, Franzosa EA, Vaarala O, de Goffau M, Harmsen H, Ilonen J, Virtanen SM, Clish CB, Oresic M, Huttenhower C, Knip M and Xavier RJ. The dynamics of the human infant gut microbiome in development and in progression toward type 1 diabetes (2015) Cell Host & Microbe 17: 260-273. Doi: 10.1016/j.chom.2015.01.001.
Ley RE, Turnbaugh PJ, Klein S and Gordon JI. Human gut microbes associated with obesity (2006) Nature 444(21): 1022-1023. Doi: 10.1038/nature4441022a.
Lovett ST. The DNA exonucleases of Escherichia coli (2011) EcoSal Plus 4(2). Doi: 10.1128/ecosalplus.4.4.7.
Mikkelsen NE, Brannvall M, Virtanen A and Kirsebom LA. Inhibition of RNAse P RNA cleavage by aminoglycosides (1999) PNAS 96: 6155-6160.
Panek M, Paljetak HC, Baresic A, Peric M, Matijasic M, Lojkic I, Bender DV, Krznaric Z and Verbanac D. Methodology challenges in studying human gut microbiota—effects of collection, storage, DNA extraction and next generation sequencing technologies (2018) Scientific Reports 8:5143. Doi: 10.1038/s41598-018-23296-4.
Pavelich IJ, Maehigashi T, Hoffer ED, Ruangprasert A, Miles SJ and Dunham CM. Monomeric YoeB toxin retains RNAse activity but adopts an obligate dimeric form for thermal stability (2019). Nucleic Acids Research 47(19): 10400-10413. Doi: 10.1093/narlgkz760.
Qin J, Li Y, Cai Z, Li S, Zhu J, Zhang F, Liang S, Zhang W, Guan Y, Shen D, Peng Y, Zhang D, Jie Z, Wu W, Qin Y, Xue W, Li J, Han L, Lu D, Wu P, Dai Y, Sun X, Li Z, Tang A, Zhong S, Li X, Chen W, Xu R, Wang M, Feng Q, Gong M, Yu J, Zhang Y, Zhang M, Hansen T, Sanchez G, Raes, J, Falony G, Okuda S, Almeida M, LeChatelier E, Renault P, Pons N, Batto J-M, Zhang Z, Chen H, Yang R, Zheng W, Li S, Yang H, Wang J, Ehrlich SD, Nielsen R, Pedersen O, Kristiansen K and Wang J. A metagenome-wide association study of gut microbiota in type 2 diabetes (2012) Nature 490: 55-60. Doi: 10.1038/nature11450.
Schaubeck M, Clavel T, Calasan J, Lagkouvardos I, Haange SB, Jehmlich N, Basic M, Dupont A, Hornef M, von Bergen M, Bleich A and Haller D. Dysbiotic gut microbiota causes transmissible Crohn's disease-like ileitis independent of failure in antimicrobial defence (2015) Gut 65: 225-237. Doi: 10.1136/gutjnl-2015-309333.
Scher JU, Sczesnak A, Longman RS, Segata N, Ubeda C, Bielski C, Rostron T, Cerundolo V, Pamer EG, Abramson SB, Huttenhower C and Littman DR. Expansion of intestinal Prevotella copri correlates with enhanced susceptibility to arthritis (2013) eLife 2: e01202. Doi: 10.7554/eLife.01202.
Schulz MD, Atay C, Heringer J, Romrig FK, Schwitalla S, Aydin B, Ziegler PK, Varga J, Reindl W, Pommerenke C, Salinas-Riester G, Bock A, Alpert C, Blaut M, Polson SC, Brandl L, Kirchner T, Greten FR, Polson SW and Arkan MC. High-fat diet-mediated dysbiosis promotes intestinal carcinogenesis independent of obesity (2014) Nature 514(7523): 508-512. Doi: 10.1038/nature13398.
Sorrentino S. The eight human “canonical” ribonucleases: Molecular diversity, catalytic properties, and special biological actions of the enzyme proteins (2010) FEBS Letters 584: 2194-2200. Doi: 10.1016/febslet.2010.04.018.
Thorn A, Steinfeld R, Ziegenbein M, Grapp M, Hsiao H-H, Urlaub H, Sheldrick GM, Gartner J and Kratzner R. Structure and activity of the only human RNAse T2 (2012) Nucleic Acids Research 40(17): 8733-8742. Doi: 10.1093/nar/gks614.
Wolf B, Lesnaw JA and Reichmann ME. A mechanism of the irreversible inactivation of bovine pancreatic ribonuclease by diethylpyrocarbonate (1970) Eur J Biochem 13: 519-525.
Wu S, Mao G and Kirsebom LA. Inhibition of bacterial RNAse P RNA by phenothiazine derivatives (2016) Biomolecules 6, 38. Doi. 10.3390/biom6030038.
Yang W. Nucleases: Diversity of structure, function and mechanism (2011) Q Rev Biophys 44(1): 1-93. Doi: 10.1017/S0033583510000181.
Yano JM, Yu K, Donaldson GP, Shastri GG, Ann P, Ma L, Nagler CR, Ismagilov RF, Mazmanian SK and Hsiao EY. Indigenous bacteria from the gut microbiota regulate host serotonin biosynthesis (2015) Cell 161: 264-276. Doi: 10.1016/j.cell.2015.02.047.
Zhang L and Reha-Krantz LJ. Nuclease (2013) Brenner's Encyclopedia of Genetics, 2nd edition, Volume 5, 118-123. Doi: 10.1016/B978-0-12-374984-0.01069-X.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1. A method of stabilizing nucleic acid contained in a biological sample at ambient temperature comprising the steps of:
a) obtaining a biological sample;
b) contacting the biological sample with an aqueous composition to form a mixture, wherein the aqueous composition comprises:
(i) a denaturing agent selected from sodium dodecyl sulphate (SDS), lithium dodecyl sulphate, or a guanidinium salt;
(ii) aurintricarboxylic acid (ATA), or a salt thereof; and
(iii) at least one of a chelating agent and a buffering agent;
wherein the composition has a pH of 4.9 or less;
c) homogenizing the mixture of (b) to form a homogeneous mixture; and
d) storing the homogeneous mixture at ambient temperature.

2. The method of claim 1, wherein the aqueous composition has a pH of from 3.8 to 4.9, or a pH of from 4.3 to 4.7.

3. The method of claim 1 or 2, wherein the denaturing agent is SDS and wherein the aqueous composition comprises a chelating agent and, optionally, a buffering agent.

4. The method of claim 3, wherein the chelating agent is selected from ethylene glycol tetraacetic acid (EGTA), (2-hydroxyethyl)ethylenediaminetriacetic acid (HEDTA), diethylene triamine pentaacetic acid (DTPA), nitrilotriacetic acid (NTA), ethylenediaminetriacetic acid (EDTA), 1,2-cyclohexanediaminetetraacetic acid (CDTA), N,N-bis(carboxymethyl)glycine, triethylenetetraamine (TETA), tetraazacyclododecanetetraacetic acid (DOTA), desferioximine, citrate anhydrous, sodium citrate, calcium citrate, ammonium citrate, ammonium bicitrate, citric acid, diammonium citrate, ferric ammonium citrate, lithium citrate, or a combination thereof.

5. The method of claim 4, wherein the chelating agent is CDTA.

6. The method of claim 4 or 5, wherein the chelating agent is present at a concentration of from about 25 mM to about 250 mM, or from about 50 mM to about 150 mM, or about 100 mM.

7. The method of any one of claims 3-6, wherein the aqueous composition further comprises an inorganic salt.

8. The method of claim 7, wherein the inorganic salt is a lithium salt or a sodium salt that is soluble in the aqueous composition.

9. The method of claim 7 or 8, wherein the inorganic salt is lithium sulphate, lithium chloride, sodium chloride, or any combination thereof.

10. The method of any one of claims 7-9, wherein the inorganic salt is present at a concentration of from about 100 mm to about 750 mM, or from about 200 mM to about 600 mM, or about 500 mM, or about 250 mM.

11. The method of any one of claims 3-10, wherein the SDS is present at a concentration of from about 2% to about 12% (w/v), or from about 3% to about 9% (w/v), or from about 4% to about 8% (w/v), or about 4% (w/v), or about 8% (w/v).

12. The method of claim 1 or 2, wherein the denaturing agent is a guanidinium salt, and wherein the aqueous composition comprises a buffering agent.

13. The method of claim 12, wherein the guanidinium salt is guanidinium thiocyanate or guanidinium hydrochloride.

14. The method of claim 13, wherein the guanidinium salt is guanidinium thiocyanate.

15. The method of claim 14, wherein the guanidinium thiocyanate is present at a concentration of from about 1 M to about 6 M, or from about 1 M to about 4 M, or from about 1.5 M to about 2.5 M, or about 2 M.

16. The method of claim 13, wherein the guanidinium salt is guanidinium hydrochloride.

17. The method of claim 16, wherein the guanidinium hydrochloride is present at a concentration of from about 1 M to about 6 M, or from about 2 M to about 5 M, or from about 3.5 M to about 4.5 M, or about 4 M.

18. The method of any one of claims 1-17, wherein the buffering agent is sodium acetate.

19. The method of any one of claims 1-18, wherein the buffering agent is present at a concentration of from about 10 mM to about 500 mM, or from about 25 mM to about 250 mM, or from about 25 mM to about 150 mM, or from about 25 mM to about 75 mM, or about 50 mM.

20. The method of any one of claims 1-19, wherein the ATA, or the salt thereof, is present at a concentration of from about 2.5 mM to about 50 mM, or from about 5 mM to about 15 mM, or about 10 mM.

21. The method of any one of claims 1-20, wherein the aqueous composition further comprises polyacrylic acid (PAAc), or a salt thereof.

22. The method of claim 21, wherein the PAAc, or the salt thereof, has a molecular weight of from about 2,000 to about 10,000, or from about 2,000 to about 5,000, or about 5,000.

23. The method of claim 21 or 22, wherein the PAAc, or the salt thereof, is present at a concentration of from about 5 mg/mL to about 20 mg/mL, or from about 5 mg/ml to about 15 mg/mL, or about 10 mg/mL.

24. The method of any one of claims 1-23, wherein the ambient temperature is from about 15° C. to about 25° C.

25. The method of any one of claims 1-24, wherein the biological sample is a saliva sample or a fecal sample.

26. The method of claim 25, wherein the biological sample is a saliva sample obtained from a mammal.

27. The method of claim 26, wherein the mammal is a human.

28. The method of claim 25, wherein the biological sample is a feces sample obtained from a mammal.

29. The method of claim 28, wherein the mammal is a human.

30. The method of any one of claims 1-29, wherein the nucleic acid is deoxyribonucleic acid (DNA).

31. The method of any one of claims 1-29, wherein the nucleic acid is ribonucleic acid (RNA).

32. The method of any one of claims 1-31, wherein the method renders the nucleic acid stable for at least 7 days at a temperature of from about 15° C. to about 25° C., or for at least 14 days at a temperature of from about 15° C. to about 25° C.

33. The method of claim 1 or 2, wherein the aqueous composition comprises, consists essentially of, or consists of:
(i) a denaturing agent selected from lithium dodecyl sulphate, SDS, or a combination thereof;
(ii) aurintricarboxylic acid (ATA), or a salt thereof;
(iii) a chelating agent;
(iv) polyacrylic acid (PAAc), or a salt thereof; and
(v) an inorganic salt, wherein the inorganic salt is a lithium salt or a sodium salt that is soluble in the aqueous composition.

34. The method of claim 33, wherein:
the denaturing agent is SDS;
the chelating agent is CDTA;
the PAAc, or the salt thereof, has a molecular weight of from about 2,000 to about 10,000, or from about 2,000 to about 5,000, or about 5,000;
the inorganic salt is lithium sulphate, lithium chloride, sodium chloride, or any combination thereof.

35. The method of claim 34, wherein:
the SDS is present at a concentration of from about 2% to about 12% (w/v), or from about 3% to about 9% (w/v), or from about 4% to about 8% (w/v), or about 4% (w/v), or about 8% (w/V);
the ATA, or the salt thereof, is present at a concentration of from about 2.5 mM to about 50 mM, or from about 5 mM to about 15 mM, or about 10 mM;
the chelating agent is present at a concentration of from about 25 mM to about 250 mM, or from about 50 mM to about 150 mM, or about 100 mM;
the PAAc, or the salt thereof, is present at a concentration of from about 5 mg/mL to about 20 mg/mL, or from about 5 mg/mL to about 15 mg/mL, or about 10 mg/mL;
the inorganic salt is present at a concentration of from about 100 mm to about 750 mM, or from about 200 mM to about 600 mM, or about 500 mM, or about 250 mM.

36. An aqueous composition for stabilizing nucleic acids contained in a biological sample at ambient temperature, comprising:
(i) a denaturing agent selected from sodium dodecyl sulphate (SDS), lithium dodecyl sulphate, or a guanidinium salt;
(ii) aurintricarboxylic acid (ATA), or a salt thereof; and
(iii) at least one of a chelating agent and a buffering agent;
wherein the composition has a pH of 4.9 or less.

37. The aqueous composition of claim 36, wherein the aqueous composition has a pH of from 3.8 to 4.9, or a pH of from 4.3 to 4.7.

38. The aqueous composition of claim 36 or 37, wherein the denaturing agent is SDS and wherein the aqueous composition comprises a chelating agent and, optionally, a buffering agent.

39. The aqueous composition of claim 38, wherein the chelating agent is selected from ethylene glycol tetraacetic acid (EGTA), (2-hydroxyethyl)ethylenediaminetriacetic acid (HEDTA), diethylene triamine pentaacetic acid (DTPA), nitrilotriacetic acid (NTA), ethylenediaminetriacetic acid (EDTA), 1,2-cyclohexanediaminetetraacetic acid (CDTA), N,N-bis(carboxymethyl)glycine, triethylenetetraamine (TETA), tetraazacyclododecanetetraacetic acid (DOTA), desferioximine, citrate anhydrous, sodium citrate, calcium citrate, ammonium citrate, ammonium bicitrate, citric acid, diammonium citrate, ferric ammonium citrate, lithium citrate, or a combination thereof.

40. The aqueous composition of claim 39, wherein the chelating agent is CDTA.

41. The aqueous composition of claim 39 or 40, wherein the chelating agent is present at a concentration of from about 25 mM to about 250 mM, or from about 50 mM to about 150 mM, or about 100 mM.

42. The aqueous composition of any one of claims 38-41, wherein the aqueous composition further comprises an inorganic salt.

43. The aqueous composition of claim 42, wherein the inorganic salt is a lithium salt or a sodium salt that is soluble in the aqueous composition.

44. The aqueous composition of claim 42 or 43, wherein the inorganic salt is lithium sulphate, lithium chloride, sodium chloride, or any combination thereof.

45. The aqueous composition of any one of claims 42-44, wherein the inorganic salt is present at a concentration of from about 100 mm to about 750 mM, or from about 200 mM to about 600 mM, or about 500 mM, or about 250 mM.

46. The aqueous composition of any one of claims 38-45, wherein the SDS is present at a concentration of from about 2% to about 12% (w/v), or from about 3% to about 9% (w/v), or from about 4% to about 8% (w/v), or about 4% (w/v), or about 8% (w/v).

47. The aqueous composition of claim 36 or 37, wherein the denaturing agent is a guanidinium salt, and wherein the aqueous composition comprises a buffering agent.

48. The aqueous composition of claim 47, wherein the guanidinium salt is guanidinium thiocyanate or guanidinium hydrochloride.

49. The aqueous composition of claim 48, wherein the guanidinium salt is guanidinium thiocyanate.

50. The aqueous composition of claim 49, wherein the guanidinium thiocyanate is present at a concentration of from about 1 M to about 6 M, or from about 1 M to about 4 M, or from about 1.5 M to about 2.5 M, or about 2 M.

51. The aqueous composition of claim 48, wherein the guanidinium salt is guanidinium hydrochloride.

52. The aqueous composition of claim 51, wherein the guanidinium hydrochloride is present at a concentration of from about 1 M to about 6 M, or from about 2 M to about 5 M, or from about 3.5 M to about 4.5 M, or about 4 M.

53. The aqueous composition of any one of claims 36-52, wherein the buffering agent is sodium acetate.

54. The aqueous composition of any one of claims 36-53, wherein the buffering agent is present at a concentration of from about 10 mM to about 500 mM, or from about 25 mM to about 250 mM, or from about 25 mM to about 150 mM, or from about 25 mM to about 75 mM, or about 50 mM.

55. The aqueous composition of any one of claims 36-54, wherein the ATA, or the salt thereof, is present at a concentration of from about 2.5 mM to about 50 mM, or from about 5 mM to about 15 mM, or about 10 mM.

56. The aqueous composition of any one of claims 36-55, wherein the aqueous composition further comprises polyacrylic acid (PAAc), or a salt thereof.

57. The aqueous composition of claim 56, wherein the PAAc, or the salt thereof, has a molecular weight of from about 2,000 to about 10,000, or from about 2,000 to about 5,000, or about 5,000.

58. The aqueous composition of claim 56 or 57, wherein the PAAc, or the salt thereof, is present at a concentration of from about 5 mg/mL to about 20 mg/mL, or from about 5 mg/mL to about 15 mg/mL, or about 10 mg/mL.

59. The aqueous composition of any one of claims 36-58, wherein the ambient temperature is from about 15° C. to about 25° C.

60. The aqueous composition of any one of claims 36-59, wherein the biological sample is a saliva sample or a fecal sample.

61. The aqueous composition of claim 60, wherein the biological sample is a saliva sample obtained from a mammal.

62. The aqueous composition of claim 61, wherein the mammal is a human.

63. The aqueous composition of claim 60, wherein the biological sample is a feces sample obtained from a mammal.

64. The aqueous composition of claim 63, wherein the mammal is a human.

65. The aqueous composition of any one of claims 36-64, wherein the nucleic acid is deoxyribonucleic acid (DNA).

66. The aqueous composition of any one of claims 36-64, wherein the nucleic acid is ribonucleic acid (RNA).

67. The aqueous composition of claim 36 or 37, wherein the aqueous composition comprises, consists essentially of, or consists of:
(i) a denaturing agent selected from lithium dodecyl sulphate, SDS, or a combination thereof;
(ii) aurintricarboxylic acid (ATA), or a salt thereof;
(iii) a chelating agent;
(iv) polyacrylic acid (PAAc), or a salt thereof; and
(v) an inorganic salt, wherein the inorganic salt is a lithium salt or a sodium salt that is soluble in the aqueous composition.

68. The aqueous composition of claim 67, wherein:
the denaturing agent is SDS;
the chelating agent is CDTA;
the PAAc, or the salt thereof, has a molecular weight of from about 2,000 to about 10,000, or from about 2,000 to about 5,000, or about 5,000; and
the inorganic salt is lithium sulphate, lithium chloride, sodium chloride, or any combination thereof.

69. The aqueous composition of claim 68, wherein:
the SDS is present at a concentration of from about 2% to about 12% (w/v), or from about 3% to about 9% (w/v), or from about 4% to about 8% (w/v), or about 4% (w/v), or about 8% (w/V);
the ATA, or the salt thereof, is present at a concentration of from about 2.5 mM to about 50 mM, or from about 5 mM to about 15 mM, or about 10 mM;
the chelating agent is present at a concentration of from about 25 mM to about 250 mM, or from about 50 mM to about 150 mM, or about 100 mM;
the PAAc, or the salt thereof, is present at a concentration of from about 5 mg/mL to about 20 mg/mL, or from about 5 mg/mL to about 15 mg/mL, or about 10 mg/mL; and
the inorganic salt is present at a concentration of from about 100 mm to about 750 mM, or from about 200 mM to about 600 mM, or about 500 mM, or about 250 mM.

70. A stabilized biological composition comprising:
the aqueous composition of any one of claims 36-69 in combination with a biological sample.

71. The stabilized biological composition of claim 70, wherein the biological sample is a saliva sample or a fecal sample, optionally wherein the biological sample is obtained from a mammal, such as a human.
